(19) United States
(12) Patent Application Publication
(10) Pub. No.: US 2026/0083613 A1
(43) Pub. Date: Mar. 26, 2026

(54) TURF TOE REHABILITATION DEVICE AND METHOD OF USING A TURF TOE REHABILITATION DEVICE

(71) Applicant: New York University, New York, NY (US)

(72) Inventors: Vikram Kapila, West Orange, NJ (US); Archit Vireshkumar Sharma, Brooklyn, NY (US); Kishan Reddy Raghunath, Brooklyn, NY (US); Konishjit Singh Bedi, Sparks, NV (US)

(21) Appl. No.: 19/338,735

(22) Filed: Sep. 24, 2025

Related U.S. Application Data
(60) Provisional application No. 63/699,130, filed on Sep. 25, 2024.

(51) Int. Cl. A61H 1/02; G16H 20/30

(52) U.S. Cl. CPC A61H 1/0266; G16H 20/30; A61H 2001/027; A61H 2201/018; A61H 2201/0196; A61H 2201/14; A61H 2201/1673; A61H 2201/5007

(57) ABSTRACT

Turf Toe is an injury that affects the mobility of an individual's big toe, most commonly affecting the metatarsophalangeal joint (MPJ). An assistive rehabilitation device facilitates several phases of rehabilitation treatment for the injury, namely an active phase and passive phase. These exercises are needed for the patient after a few weeks of recovery. The device provides the assistive as well as resistive forces needed for the process of rehabilitation helping the patient to recover faster and carry out exercises in a structured and right way.

### § 1. CROSS-REFERENCE TO RELATED APPLICATIONS

This application claims priority to U.S. Provisional Application No. 63/699,130 filed on Sep. 25, 2024, incorporated herein by reference in its entirety.

### § 2. BACKGROUND OF THE INVENTION

### § 2.1 Field of the Invention

The present application concerns devices and methods for physical therapy. In particular, the present application concerns devices and methods for rehabilitating turf toe and the like.

### § 2.2 Background Information

American football is not only one of the most popular sports in the United States, but also one of the most physically intensive and involves repetitive high-intensity actions, and immense physical collisions. Although often associated with American football, Turf Toe occurs primarily in athletic environments, such as when an athlete pushes off to sprint or is tackled with the front of the foot fixed and jammed into the ground, causing the toe to get stuck or caught in a hyperextended position

Most turf toe injuries do not require surgery and are treated with physical therapy. Although the treatment depends on the severity of the injury, and there is a need for an individualized treatment program specific to the exact nature of an individual's condition and goals, there are some basic exercises that are a part of most lines of treatment. It would be useful to provide a device that can provide help in regaining motion using simple physical therapist-approved exercises.

The first few weeks after the injury involves putting less stress on the toe, which doesn't mean complete rest or R.I.C.E. (Rest, Ice, Compression, and Elevation), but means limiting the stress on it. This can be done by Manual Therapy, which involves gently moving and manipulating muscles and joints to improve their motion and strength. In a first phase of turf toe rehabilitation, gentle stretching exercises help regain motion.

A second phase of turf toe rehabilitation increases the stresses that are slowly applied to the tissues, which means working into some degree of big toe extension. In this second phase, it would be useful to enable the patient to apply varying pressure on the toe in a particular direction and count the number of repetitions for the same to facilitate a routine for rehabilitation exercises. It is important that the patient perform these exercises in a controlled method, to avoid hyperextension of the big toe, which is often possible when these exercises are done by a patient on their own without any controlling or limiting device or mechanism.

### § 2.2 Literature Review

Bowers and Martin originally defined “Turf Toe” injury (metatarsophalangeal joint sprain) in 1976 as a hyperextension moment of the hallux metatarsophalangeal (MTP) joint. (See, e.g., the document, Bowers K. D. Jr, Martin R. B., “Turf-toe: a shoe-surface related football injury.” Med Sci Sports 1976; 8(2):81-3, incorporated herein by reference.) This was after they observed that the University of West Virginia's football players suffered on average, 5.4 turf toe injuries per season. (See, e.g., the document, McCormick J. J., Anderson R. B., “Turf Toe: Anatomy, Diagnosis, and Treatment,” Sports Health. 2010; 2(6):487-494. doi: 10.1177/1941738110386681, incorporated herein by reference.) Typically, turf toe injuries are a result of axial load being delivered onto the foot when the upward motion of the ankle is limited. Due to the limited motion of the ankle, the axial load drives the hallux MTP into hyperextension during dorsiflexion, attenuating the plantar joint complex. (See, e.g., the document, McCormick J. J., Anderson R. B., “Turf Toe: Anatomy, Diagnosis, and Treatment,” Sports Health. 2010; 2(6):487-494. doi: 10.1177/1941738110386681, incorporated herein by reference.)

During normal gait, the capsular ligamentous complex of the hallux MTP needs to withstand 40%-60% of the body weight, which increases to 200%-300% of the body weight depending on the type of physical activity being performed. (See, e.g., the document, Stokes I. A., Hutton W. C., Stott J. R., et al., “Forces under the hallux valgus foot before and after surgery,” Clin Orthop Relat Res 1979; 142:64-72, incorporated herein by reference.) In the case of running jumps as often experienced in football games, the forces can reach up to 800% of the body weight (See, e.g., the document, Nigg B. M., Ed., “Biomechanical aspects of running,” Biomechanics of running shoes, Champaign (IL): Human Kinetics Publishers; 1986. p. 1-25, incorporated herein by reference.), exerting a considerable amount of strain on the capsular ligamentous complex.

It is important to analyze the range of motion of the toe of a control group as well as of a demographic with hallux limitation. Munuera et al conducted a study where they assessed the mobility of the first metatarsophalangeal joint (MPJ) between participants with and without hallux limitus (limited first metatarsophalangeal joint dorsiflexion). (See, e.g., the document, Munuera, P. V., Trujillo, P., & Güiza, I., “Hallux interphalangeal joint range of motion in feet with and without limited first metatarsophalangeal joint dorsiflexion,” Journal of the American Podiatric Medical Association, 102(1), 47-53, incorporated herein by reference.) Table 1, extracted from their study shows the range of motion, in degrees, of dorsiflexion and plantar flexion of MPJ.

TABLE 1
Range of Motion of first MPJ of control and hallux limitus group

| Variable and Group | Mean ± SD | 95% CI | P Value | Cohen d |
|---|---|---|---|---|
| First MPJ | | | <.001 | 4.156 |
| dorsiflexion (°) | | | | |
| Control | 69.63 ± 3.48 | 68.73-70.53 | | |
| Hallux limitus | 42.12 ± 8.69 | 39.87-44.36 | | |
| First MPJ | | | 0.001 | 0.611 |
| plantarflexion (°) | | | | |
| Control | 50.07 ± 11.85 | 47.00-53.13 | | |
| Hallux limitus | 43.60 ± 9.14 | 41.24-45.96 | | |

Abbreviations: CI, confidence interval; IPJ, interphalangeal joint; MPJ, metatarsophalangeal joint.

### § 2.3 Unmet Needs

From the findings of the Munuera study cited above, a device that can facilitate MPJ dorsiflexion and plantarflexion of 70.53° and 53.13°, respectively, would be useful. It is important to note that these are just suggested limits for the range of motion of a healthy toe and the actual range of motion of individuals using the device will have a limited range of motion. The device (and protocols for its use) should facilitate the two phases of turf toe rehabilitation, in a safe manner that avoids reinjury or further injury.

### § 3. SUMMARY OF THE INVENTION

Aspects of the present disclosure relate to a method for assisting physical therapy for an injured toe of a foot of a human, using a device having a footrest, a toe platform, and at least one motor and at least one guide for providing at least one of powered pitch and/or yaw motions of the toe platform relative to the footrest, and resistance to pitch and/or yaw motions of the toe platform relative to the footrest, the method comprising receiving and holding the foot of the human by the footrest, such that the injured toe rests of the toe platform, and retaining the injured toe on the toe platform, controlling the at least one motor to provide repeated, powered pitch and/or yaw motions of the toe platform relative to the footrest such that at least a toe of the foot of the human moves passively under external power, and controlling the at least one motor to provide repeated resistance to pitch and/or yaw motions of the toe platform relative to the footrest such that the toe of the foot of the human moves actively under externally activated resistance.

In some embodiments, the method further includes, after controlling the at least one motor to provide repeated, powered pitch and/or yaw motions of the toe platform relative to the footrest but before controlling the at least one motor to provide repeated resistance to pitch and/or yaw motions of the toe platform relative to the footrest, removing the foot of the human from the footrest and the injured toe from the toe platform, after waiting for at least a prescribed rest period between passive and active movements of toe, as prescribed by a therapist based on injury and therapy protocol, receiving and holding the foot of the human by the footrest such that the injured toe rests on the toe platform, and retaining the injured toe on the toe platform.

In some embodiments, the act of controlling the at least one motor to provide repeated, powered pitch and/or yaw motions of the toe platform relative to the footrest, limits the pitch and/or yaw motions to angular limits. In some embodiments, the angular limits are increased over time, but such that the limits are constrained to be within the normative range of pitch and/or yaw motion of human toe. In some embodiments, the angular limits are adjusted based on at least one of express patient feedback, implied patient feedback, recovery history of patient, reinjury, and rehabilitation setback. In some embodiments, the patient can lower, via a user interface, at least one of the angular limits and angular speed of pitch and yaw movements.

In some embodiments, the act of controlling the at least one motor to provide repeated resistance to at least one of pitch and yaw motions of the toe platform relative to the footrest, increases the resistance over time, and wherein the resistance is increased over time by adapting the torque resistance offered by the at least one motor.

In some embodiments, the method further includes tracking and storing at least one of a count of the repeated, powered pitch motions of the toe platform relative to the footrest, a count of the repeated, powered yaw motions of the toe platform relative to the footrest, an angular range of at least one of the repeated, powered pitch motions of the toe platform relative to the footrest, an angular range of at least one of the repeated, powered yaw motions of the toe platform relative to the footrest, a count of the repeated resistance to pitch motions of the toe platform relative to the footrest, a count of the repeated resistance to yaw motions of the toe platform, a force and/or torque of at least one of the repeated resistance to pitch motions of the toe platform relative to the footrest, a force and/or torque of at least one of the repeated resistance to yaw motions of the toe platform, an angular range of at least one of the repeated resistance to pitch motions of the toe platform relative to the footrest, and an angular range of at least one of the repeated resistance to yaw motions of the toe platform.

In some embodiments, the method further includes processing at least a portion of the tracked and stored data using one or more artificial intelligence (AI) and/or machine learning (ML) models trained to identify subject-specific rehabilitation parameters, and adjusting at least one of a powered motion or a resistance profile of the toe platform in real time based on output from the AI and/or ML models to personalize treatment for the injured toe.

Aspects of the present disclosure relate to a device for assisting physical therapy for an injured toe of a foot of a human, the device including a footrest, a toe platform, a motorized assembly mechanically coupled with the toe platform, guide rails, and a controller configured to control the motorized assembly to provide, in cooperation with the guide rails, at least one of powered pitch and/or yaw motions, either individually or simultaneously, of the toe platform relative to the footrest, and resistance to pitch and/or yaw motions, either individually or simultaneously, of the toe platform relative to the footrest.

In some embodiments, the device further includes a universal joint mechanically coupling the footrest and the toe platform. In some embodiments, the motorized assembly includes a first motor for providing, via a gear train, at least one of powered pitch motions of the toe platform relative to the footrest, and resistance to pitch motions of the toe platform relative to the footrest, and a second motor for providing at least one of powered yaw motions of the toe platform relative to the footrest, and resistance to yaw motions of the toe platform relative to the footrest.

In some embodiments, the controller controls each of the first and second motors to limit the pitch and/or yaw motions to angular limits, either individually or simultaneously. In some embodiments, the angular limits are increased over time, but such that the limits are constrained to be within the normative range of pitch and/or yaw motion of human toe.

In some embodiments, the device further includes a physical or soft button for receiving therapist and/or patient feedback, wherein the means for receiving therapist and/or patient feedback provides the feedback to the controller to control at least one of range of motion of yaw angle, range of motion of pitch angle, resistance in the yaw direction, and/or resistance in the pitch direction.

In some embodiments, the controller is further configured to track and store at least one of: a count of the repeated, powered pitch motions of the toe platform relative to the footrest, a count of the repeated, powered yaw motions of the toe platform relative to the footrest, an angular range of at least one of the repeated, powered pitch motions of the toe platform relative to the footrest, an angular range of at least one of the repeated, powered yaw motions of the toe platform relative to the footrest, a count of the repeated resistance to pitch motions of the toe platform relative to the footrest, a count of the repeated resistance to yaw motions of the toe platform, a force of at least one of the repeated resistance to pitch motions of the toe platform relative to the footrest, a force of at least one of the repeated resistance to yaw motions of the toe platform, an angular range of at least one of the repeated resistance to pitch motions of the toe platform relative to the footrest, and/or an angular range of at least one of the repeated resistance to yaw motions of the toe platform.

In some embodiments, information tracked by the controller is stored on at least one of a non transitory computer readable storage medium on the device, and/or a non transitory computer readable storage medium on a mobile device in wireless communication with the device. In some embodiments, the controller is further configured to receive a user input selecting a first or second phase of physical therapy, and select a first phase controlling the at least one motor to provide repeated, powered pitch and/or yaw motions of the toe platform relative to the footrest responsive to receiving a user input selection of the first phase of physical therapy, or select a second phase controlling the at least one motor to provide repeated resistance to pitch and/or yaw motions of the toe platform relative to the footrest responsive to receiving a user input selection of the second phase of physical therapy.

In some embodiments, the pitch has a maximum value of either approximately 35 degrees or approximately-35 degrees. In some embodiments, a range of motion of the yaw has a maximum value of approximately 30 degrees.

### § 4. BRIEF DESCRIPTION OF THE DRAWINGS

FIG. 1 is a perspective view of the exterior of an example turf toe rehabilitation device consistent with the present description.

FIG. 2 is a front view of the exterior of an example turf toe rehabilitation device consistent with the present description.

FIG. 3 is a top view of the exterior of an example turf toe rehabilitation device consistent with the present description.

FIG. 4 is a bottom view of the exterior of an example turf toe rehabilitation device consistent with the present description.

FIG. 5 is a right-side view of the exterior of an example turf toe rehabilitation device consistent with the present description.

FIG. 6 is a left-side view of the exterior of an example turf toe rehabilitation device consistent with the present description.

FIG. 7 is a perspective view of the interior of an example turf toe rehabilitation device consistent with the present description.

FIG. 8 is a front view of the interior of an example turf toe rehabilitation device consistent with the present description.

FIG. 9 is a top view of the interior of an example turf toe rehabilitation device consistent with the present description.

FIG. 10 is a right-side view of the interior of an example turf toe rehabilitation device consistent with the present description.

FIG. 11A is a left-side view of the interior of an example turf toe rehabilitation device consistent with the present description. FIG. 11B is a perspective cut-away view of an example turf toe rehabilitation device. FIG. 11C is a side cut-away view of an example turf toe rehabilitation device including details of a second guide rail(s).

FIG. 12 is a perspective view of an example motorized platform assembly of an example turf toe rehabilitation device consistent with the present description.

FIG. 13A is a front view of the example motorized platform assembly of an example turf toe rehabilitation device consistent with the present description. FIG. 13B is a front cut-away view of the example motorized platform assembly within the example turf toe rehabilitation device.

FIG. 14 is a top view of the example motorized platform assembly of an example turf toe rehabilitation device consistent with the present description.

FIG. 15 is a right-side view of the example motorized platform assembly of an example turf toe rehabilitation device consistent with the present description.

FIG. 16 is a left-side view of the example motorized platform assembly of an example turf toe rehabilitation device consistent with the present description.

FIG. 17A is a perspective view of an example toe platform assembly of an example turf toe rehabilitation device consistent with the present description. FIG. 17B is a cut-away perspective view of the example toe platform assembly as attached to the example turf toe rehabilitation device.

FIG. 18 is a top view of the example toe platform assembly of an example turf toe rehabilitation device consistent with the present description.

FIG. 19 is a front view of the example toe platform assembly of an example turf toe rehabilitation device consistent with the present description.

FIG. 20 is a left-side view of the example toe platform assembly of an example turf toe rehabilitation device consistent with the present description.

FIG. 21 is a flow diagram of an example method for controlling an example turf toe rehabilitee device consistent with the present description.

FIG. 22 is a flow diagram of an example method for assisting physical therapy for an injured toe of a foot of a human, using an example turf toe rehabilitee device consistent with the present description.

FIG. 23 illustrates an example device (e.g., computer) that may be used to perform one or more of the processes described, and/or store information used and/or generated by such processes.

FIGS. 24-28 illustrate example user interface screens consistent with the present description.

### § 5. DETAILED DESCRIPTION

Example embodiments consistent with the present description may involve novel methods, apparatus, message formats, data structures, and/or user interfaces for helping a patient and/or therapist rehabilitate turf toe. The following description is presented to enable one skilled in the art to make and use the invention, and is provided in the context of particular applications and their requirements. Thus, the following description of embodiments consistent with the present description provides illustration and description, but is not intended to be exhaustive or to limit the present invention to the precise form disclosed. Various modifications to the disclosed embodiments will be apparent to those skilled in the art, and the general principles set forth below may be applied to other embodiments and applications. For example, although a series of acts may be described with reference to a flow diagram, the order of acts may differ in other embodiments when the performance of one act is not dependent on the completion of another act. Further, non-dependent acts may be performed in parallel. No element, act or instruction used in the description should be construed as critical or essential to the present invention unless explicitly described as such.

Unless otherwise specifically defined herein, all terms are to be given their broadest reasonable interpretation. This includes meanings implied from the specification as well as meanings understood by those skilled in the art and/or as defined in dictionaries, treatises, etc.

It is noted that, as used in this specification and the appended claims, the singular forms “a,” “an,” and “the” include plural referents unless otherwise specified. The term “includes” and/or “including,” when used in this specification, specify the presence of stated features, elements, and/or components, but do not preclude the presence or addition of one or more other features, steps, operations, elements, components, and/or groups thereof.

Relative terms such as “horizontal,” “vertical,” “up,” “down,” “top,” and “bottom” as well as derivatives thereof (e.g., “horizontally,” “downwardly,” “upwardly,” etc.) should be construed to refer to the orientation as then-described or as shown in the drawing figure under discussion. These relative terms are for convenience of description and normally are not intended to require a particular orientation in actuality. Terms including “inwardly” versus “outwardly,” “longitudinal” versus “lateral,” and the like are to be interpreted relative to one another or relative to an axis of elongation, or an axis or center of rotation, as appropriate. Terms concerning attachments, coupling and the like, such as “connected” and “interconnected,” refer to a relationship wherein structures are secured or attached to one another either directly or indirectly through intervening structures, as well as both movable or rigid attachments or relationships, unless expressly described otherwise. The phrases “operatively” or “operably connected” indicates such an attachment, coupling, or connection that allows the pertinent structures to operate as intended by virtue of that relationship.

Reference throughout the specification to “one embodiment,” “an embodiment,” or “some embodiments” means that a particular feature, structure, or characteristic described in connection with at least one embodiment of the subject matter is included in at least one embodiment of the subject matter disclosed. Thus, the appearance of the phrases “in one embodiment,” “in an embodiment,” or “in some embodiments” in various places throughout the specification is not necessarily referring to the same embodiment. Further, the particular features, structures, or characteristics of “one embodiment,” “an embodiment,” or “some embodiments” may be combined in any suitable manner with each other to form additional embodiments of such combinations. It is intended that embodiments of the disclosed subject matter cover modifications and variations thereof. Terms such as “first,” “second,” “third,” etc., merely identify one of a number of portions, components, steps, operations, functions, and/or points of reference as disclosed herein, and likewise to not necessarily limit embodiments of the present disclosure to any particular configuration or orientation.

Moreover, throughout this disclosure, various aspects can be presented in a range format. It should be understood that the description in range format is merely for convenience and brevity and should not be construed as an inflexible limitation on the scope of the disclosure. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6, etc., as well as individual numbers within that range, for example, 1, 2, 2.7, 3, 4, 5, 5.3, 6, and any whole and partial increments therebetween. This applies regardless of the breadth of the range. As used herein, the term “about” in reference to a measurable value, such as an amount, a temporal duration, and the like, is meant to encompass the specified value or variations of plus or minus 20%, plus or minus 10%, plus or minus 5%, plus or minus 1%, and plus or minus 0.1% of the specified value, as such variations are appropriate and fit within the confines of a functional system.

The terms “proximal,” “distal,” “anterior,” “posterior,” “medial,” “lateral,” “superior,” and “inferior” are defined by their standard usage indicating a directional term of reference. For example, “proximal” refers to a position that is situated nearer to the center of a body or point of attachment or interest. In another example, “anterior” refers to the front of a body or structure, while “posterior” refers to the rear of a body or structure, in relation to a relative viewpoint. In another example, “medial” refers to the direction towards the midline of a body or structure, and “lateral” refers to the direction away from the midline of a body or structure. In some examples, “lateral” or “laterally” may refer to any sideways direction. In another example, “superior” refers to the top of a body or structure, while “inferior” refers to the bottom of a body or structure. It should be understood, however, that the directional term of reference may be interpreted within the context of a specific body or structure, such that a directional term referring to a location in the context of the reference body or structure may remain consistent as the orientation of the body or structure changes.

The terms “patient,” “subject,” “individual,” “user”, and the like are used interchangeably herein, and refer to any animal amenable to the systems, devices, and methods described herein. The patient, subject, individual, or user may be a mammal, and in some instances, a human.

### § 5.1 EXAMPLE DEVICE(S)

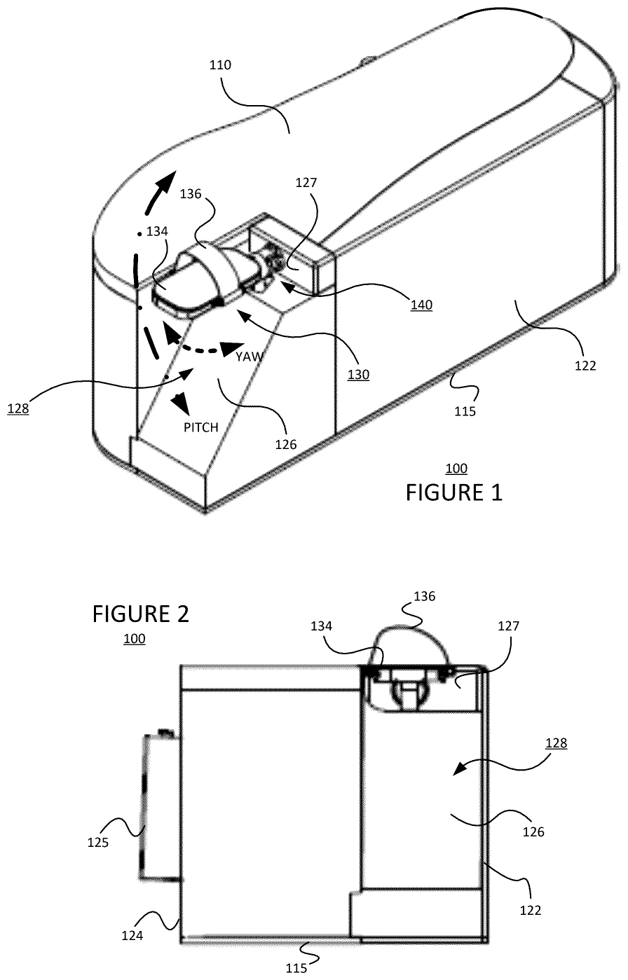

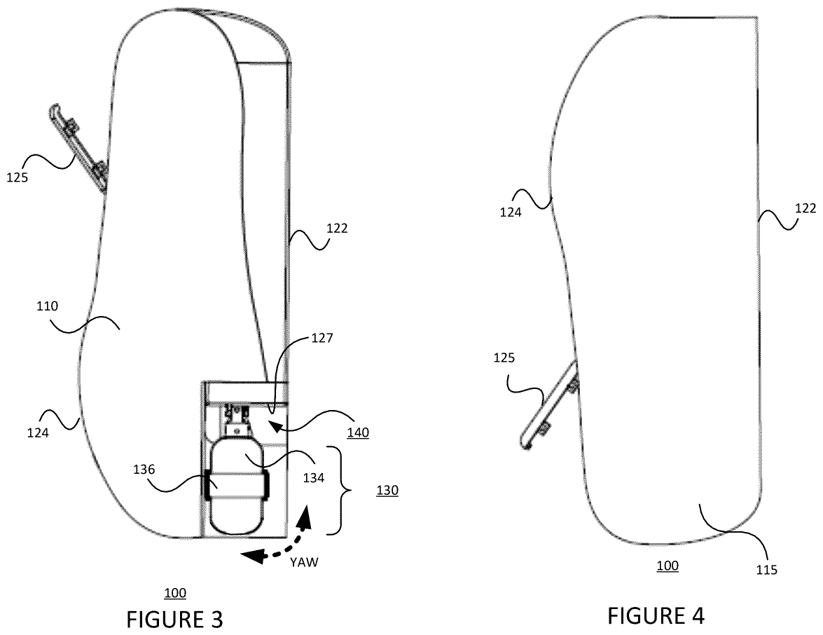

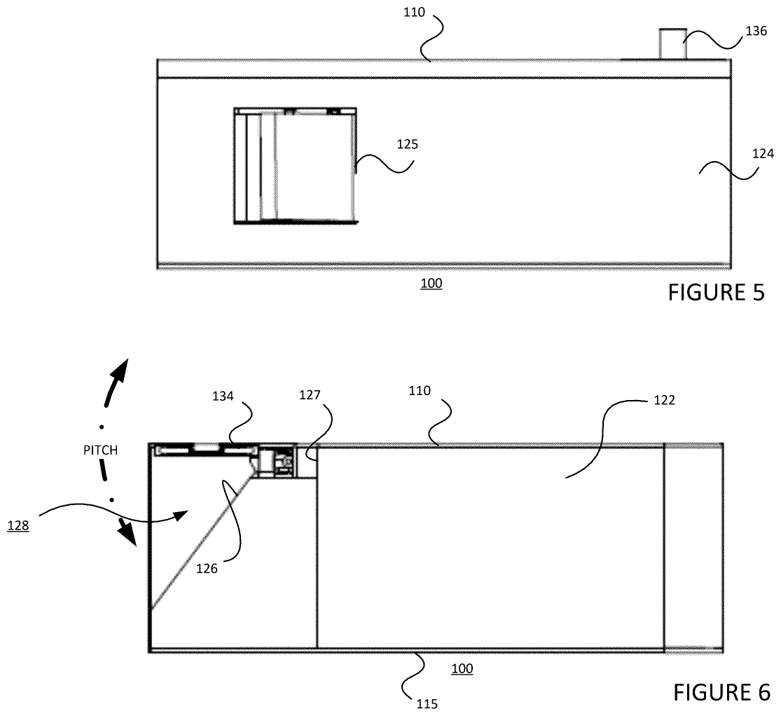

FIGS. 1-6 are perspective, front, top, bottom, right-side and left-side views, respectively, of the exterior of an example turf toe rehabilitation device 100 consistent with the present description. Referring to FIGS. 1-6 collectively, the example device 100 includes a footbed 110, a base plate 115, a toe platform assembly 130 being mechanically coupled with a joint having two degrees of freedom (e.g., a universal joint 140), which allows the toe platform assembly 130 to have pitch and yaw rotations (see dot-dashed arc and dashed arc, respectively.) relative to the footbed 110. Generally, the toe platform assembly 130 comprises a toe platform 134 and a strap 136. A left side wall 122 and a right side wall 124 are arranged between the base plate 115 and the footbed 110, and support the footbed 110. Referring to FIGS. 1, 2 and 6, an inclined surface 126 defines a space 128 allowing for pitch rotation of the toe platform 134. Referring to FIGS. 2 and 3, the space 128 (e.g., an open space) allows for yaw rotation of the toe platform 134 outward from the footbed 110. Referring to FIGS. 3, 4 and 5, a battery compartment access door 125 is provided on the right side wall 124 of the example device 100, however the position of the door 125 may be anywhere on the device, such as provided on left side wall 122, base plate 115, or footbed 110.

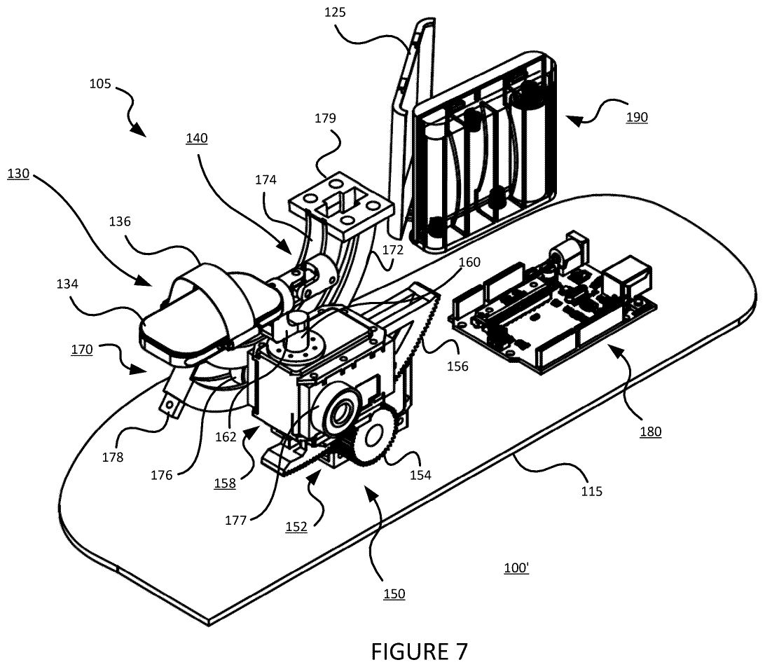

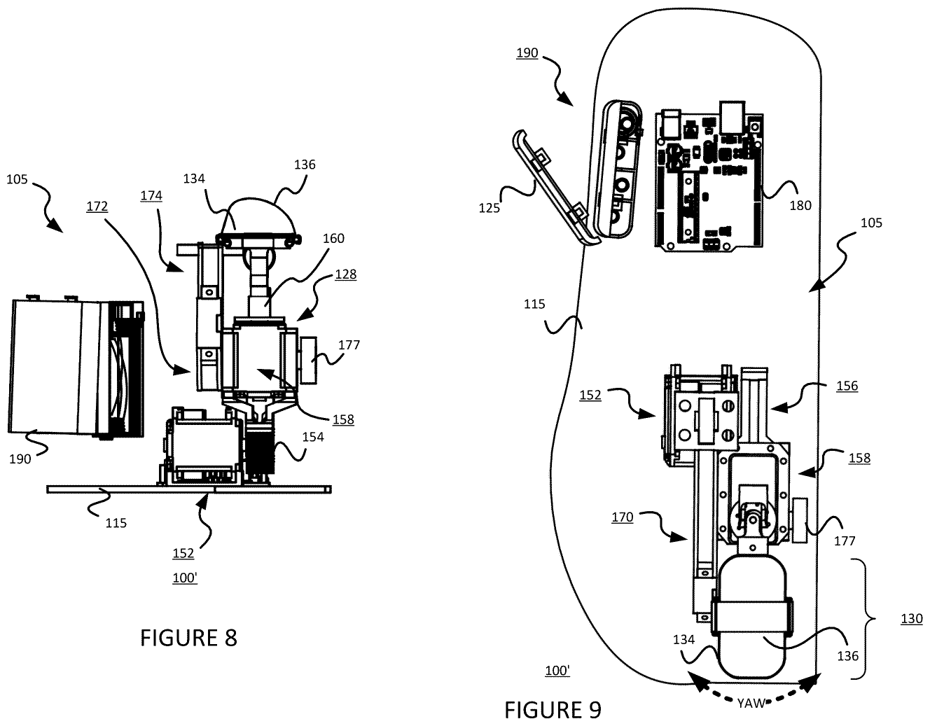

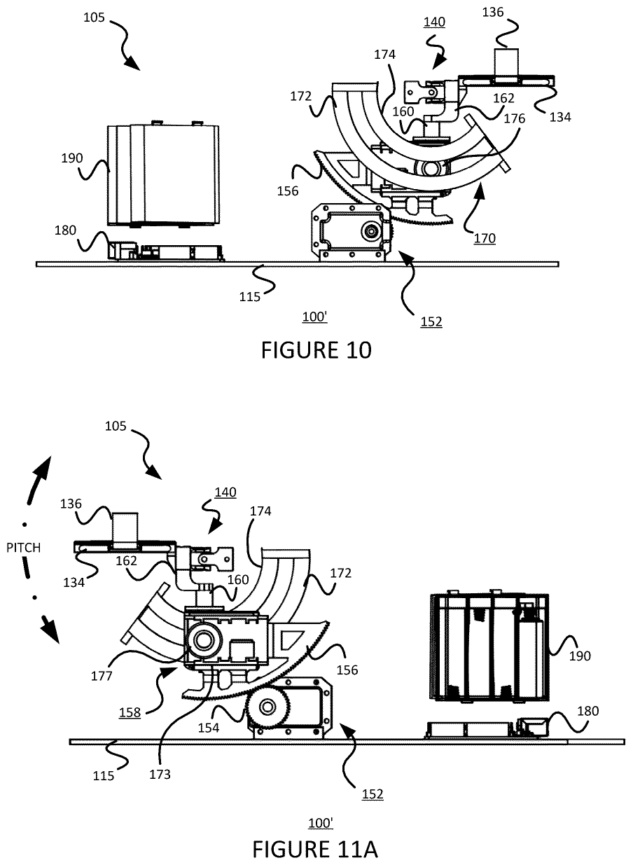

FIGS. 7-11A are perspective, front, top, right-side, and left-side views, respectively, of the interior 105 of an example turf toe rehabilitation device 100′ consistent with the present description. As shown, the interior 105 includes a motorized platform assembly 150, a microcontroller 180, and a battery compartment 190. The microcontroller 180 can be used to control movements of the motorized platform assembly 150 and perform example turf toe rehabilitation methods, as described herein. The motorized platform assembly 150 is provided to allow, and/or to drive, pitch and yaw rotations of the toe platform 134 relative to the footbed 110. Finally, the battery compartment access door (e.g., a cover) 125 permits a user to replace batteries in the battery compartment 190.

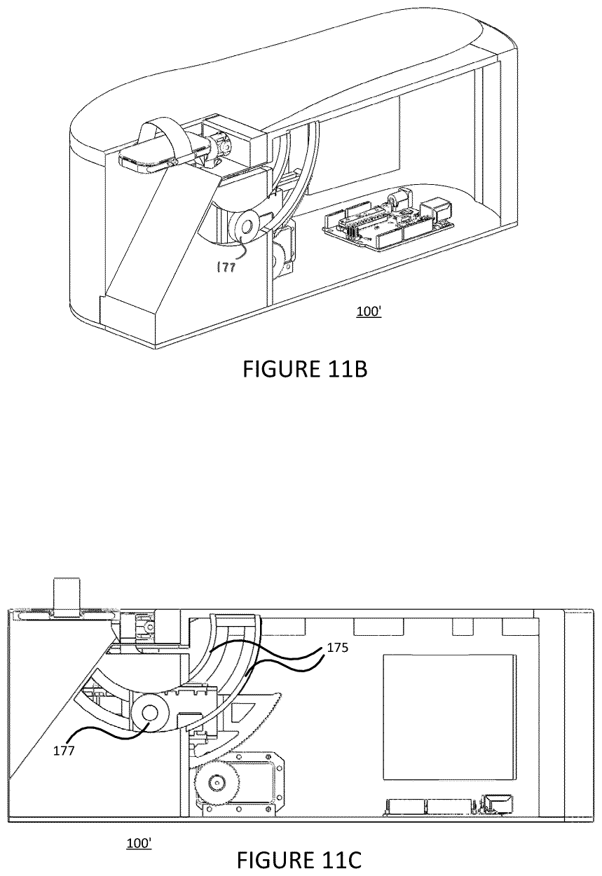

The example motorized platform assembly 150 includes a first (e.g., base/fixed) servomotor 152, a first spur gear 154 (e.g., a small gear, a pinion), a second spur gear 156 (e.g. a large and/or partial gear, a rack, a curved rack, a spur gear rack), a second servomotor 158 having a shaft 160, a bent arm 162, the toe platform assembly 130 (which includes the toe platform 134 and the toe strap 136), a universal joint 140 and a guide assembly 170. In one example embodiment, the guide assembly 170 includes a lower guide rail 172 and an upper guide rail 174, each (or at least one) having a front stop 178 and a rear stop 179. The second servomotor 158 is fitted with (or is held by a bracket fitted with) a first (e.g., right) bearing 176 and a second (e.g., left) bearing 177. The first bearing 176 can travel along an arc defined by the lower guide rail 172 and the upper guide rail 174. The second bearing 176 can travel along an arc defined by additional guide rails 175. As shown in FIGS. 11B and 11C, the additional guide rails 175 can be attached to (e.g., be integrally formed with) the inside of the left side wall 122.

The example turf toe rehabilitation device 100/100′ features a sophisticated mechanism for precise movement of a toe platform, enabling controlled pitch and yaw motions to treat turf-toe injuries. This functionality is achieved by integrating the two servomotors 152 and 158, each dedicated to one axis of movement; the first servomotor 152 provided for pitch rotation, and the second servomotor 158 provided for yaw rotation. The servomotors 152, 158 and their corresponding rotations are controlled using the microcontroller 180. The servomotors 152, 158 and the microcontroller may be powered by any power source (e.g., batteries). In some example embodiments, the microcontroller 180 can be accessed (e.g., via a wired or wireless interface) through any computing device with an interface (e.g., smartphone application).

Referring to FIGS. 7-11C the first servomotor 152 is firmly mounted on the base plate 115, and is used to drive a first (e.g., small, circular) spur gear 154 that is mounted on the drive shaft of first servomotor 152. The first spur gear 154, in turn, drives a second (e.g., large, partial) spur gear 156. The center of rotation of the second spur gear 156 coincides with a horizontal pin 148 (described later) in the universal joint 140. The second spur gear 156 supports the second servomotor 158. The second spur gear 156 has boundary stops (not shown) at both ends to ensure that it remains engaged with the first spur gear 154. Alternatively, or in addition, in some example embodiments, the microcontroller 180 is programmed to limit the rotation of first spur gear 154 (and therefore, to limit the rotation of the second spur gear 156). Alternatively, or in addition, in some example embodiments, physical stops 178 and 179 in the lower and upper guide rails 172 and 174 ensure that first and second spur gears 154 and 156 do not disengage.

As the first servomotor 152 drives the first spur gear 154 which, in turn, drives the second spur gear 156, the first (e.g., right) bearing 176 of the second servomotor 158 rides along a path defined by the lower and upper guide rails 172 and 174. Similarly, the second (e.g., left) bearing 177 of the second servomotor 158 rides along a path defined by additional guide rails (not shown). This imparts a pitch rotation of the toe platform 134 relative to the footbed 110.

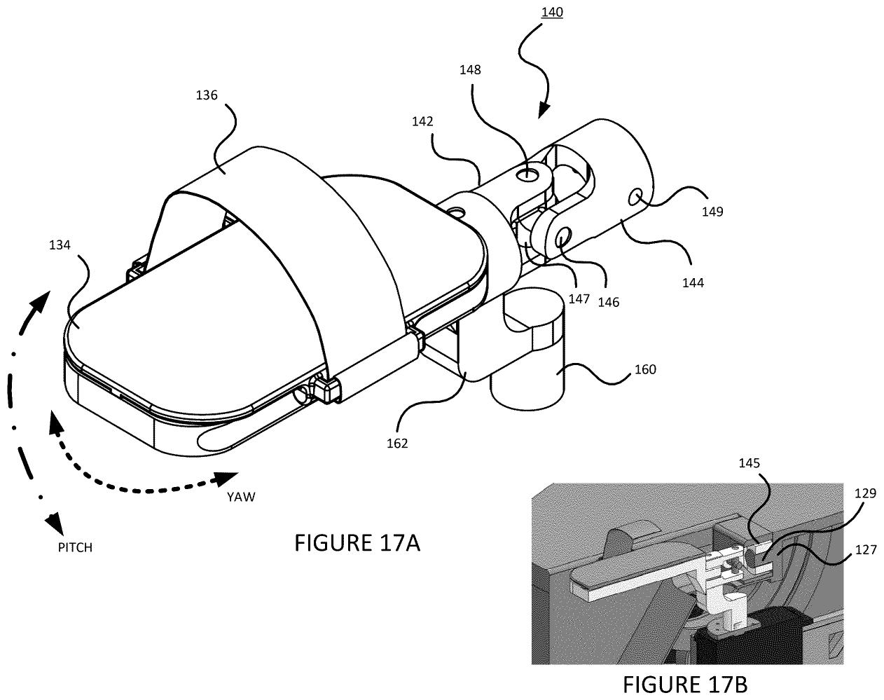

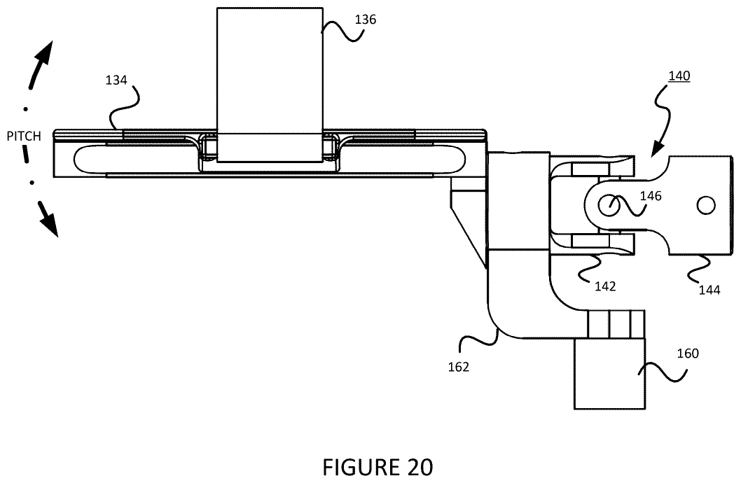

A drive shaft 160 of the second servomotor 158 is connected to a bent arm 162. The bent arm 162 hosts the toe platform assembly 130. FIGS. 17A-20 are perspective, another perspective, top, front, and left-side views, respectively, of an example toe platform assembly 130 (among other components) of an example turf toe rehabilitation device 100 consistent with the present description. As shown, this toe platform assembly 130 is coupled with a universal joint 140. The universal joint includes a yoke/fork extension 142 extending from a rear portion of the toe platform 134, a yoke/fork attachment 144 attached to the vertical wall 127 (Recall, for example, FIGS. 1-3), a horizontal pin 146, a vertical pin 148, and a center block 147. Note that a spider assembly may be used instead of the center block 147 and pins 146, 148. The second servomotor 158 can be used to impart a yaw rotation to the toe platform 134 via the drive shaft 160, the bent arm 162 and the yoke/fork extension 142. The yoke/fork attachment 144 is connected to a pin 149 passing through a mounting hole 145. The pin 149 holds the yoke/fork attachment 144 to the wall 127 and is configured to allow yoke/fork attachment 144 to rotate within mounting hole 145. As shown in FIG. 17B, the wall 127 may include a projection 129 extending into a void defined by the yoke/fork attachment 144. In some example embodiments, yoke/fork attachment 144 is fixedly and/or rigidly attached to at least one of wall 127 and projection 129. In some example embodiments, instead of, or in addition to a pin 149, yoke/fork attachment 144 may comprise a bearing (not shown) positioned between the void of the yoke/fork attachment 144 and projection 129 that enables smooth rotation and prevents forward translation of yoke/fork attachment 144 and/or the pulling out of yolk/fork attachment off of projection 129. The bearing (not shown) may be affixed to either of the yoke/fork attachment 144 with a friction/compression fit and/or other mechanical means such as with a tongue and groove interface and/or with an adhesive.

The microcontroller 180 hosts the firmware and/or runs the software to monitor and control the first and second servomotors 152,158. The battery compartment 190 holds the battery (ies) that supply power to the microcontroller 180 and the first and second servomotors 152,158. The cover or door 125 of the battery compartment 190 can be opened to access and remove the batteries for replacement (or for recharging).

As described in detail below, the up-down motion (the pitch rotation) of the toe platform 134 (e.g. big toe platform) is delivered by the first servomotor 152, and the side-to-side motion (the yaw rotation) of the toe platform 134 is delivered by the second servomotor 158. More specifically, the first servomotor 152 is mounted to the base plate 115. The first servomotor 152 is responsible for actuating the toe platform 134 up and down. The first servomotor 152 operates through a gear train, utilizing the set of spur gears 154, 156. The first spur gear 154 is attached to the shaft of the first servomotor 152, and meshes with the second (e.g., partial) spur gear 156. The second spur gear 156 is (e.g., directly) affixed to the second servomotor 158. This arrangement allows the first servomotor 152 to move the entire assembly, including the second servo motor 158, within the upper and lower guide rails 172, 174 (and the additional guide rails (not shown)), thereby moving the toe platform 134 in the pitch direction.

Recall that the first (e.g., right) bearing 176 moves along an arc defined by the upper and lower guide rails 172, 174, and that the second (e.g., left) bearing 177 moves along an arc defined by additional guide rails (not shown). More specifically, during the pitch motion, the movement of the second servomotor 158 assembly is guided by two parallel guide rails. The lower and upper guide rails 172, 174 may be mounted to the roof of the enclosure (that is, the bottom surface of the footbed 110). The corresponding additional guide rails (now shown) may be embedded into the device's main body (e.g., on the inside of left side wall 122). The upper and lower guide rails 172, 174 and the additional guide rails (now shown) define an arc that provides a stable and guided pathway for smooth, accurate pitch motion, thereby ensuring structural rigidity and consistent performance during use. The added stability provided by these guide rails reduce (e.g., prevent) any unwanted wobbling or misalignment during operations using a pitch motion.

The second (partial) spur gear 156 helps to control the range of pitch movement. In some example embodiments, the second spur gear 156 is designed with deliberate stops at its extremes so that when the toe platform 134 reaches its upper or lower pitch limit, the second (partial) spur gear 156 contacts the device's enclosure (that is, the lower surface of the footbed 110 or the upper surface of the base plate 115), acting as a mechanical stop. Bumpers (not shown) made of rubber, silicon, or the like, can be used to buffer any such contact. This ensures that the toe platform 134 stays within its designated range of motion, preventing overextension and providing a built-in safety feature and program limit. Alternatively, or in addition, stops in the guide rails can be used to limit pitch motion. Alternatively, or in addition, pitch motion can be limited by limiting the rotation of the first servomotor 152.

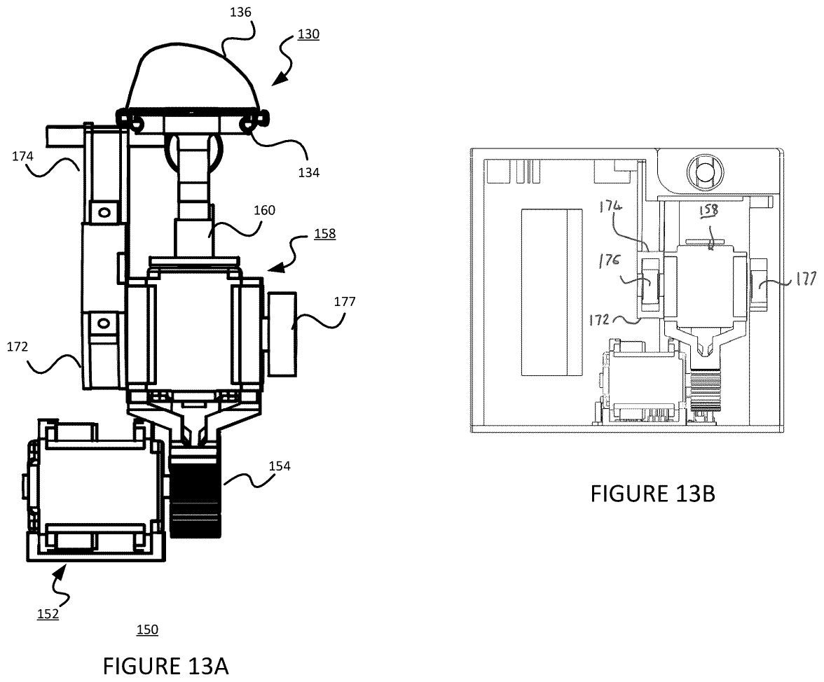

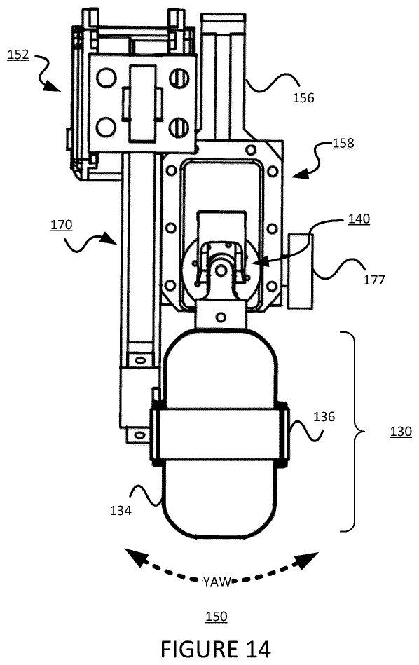

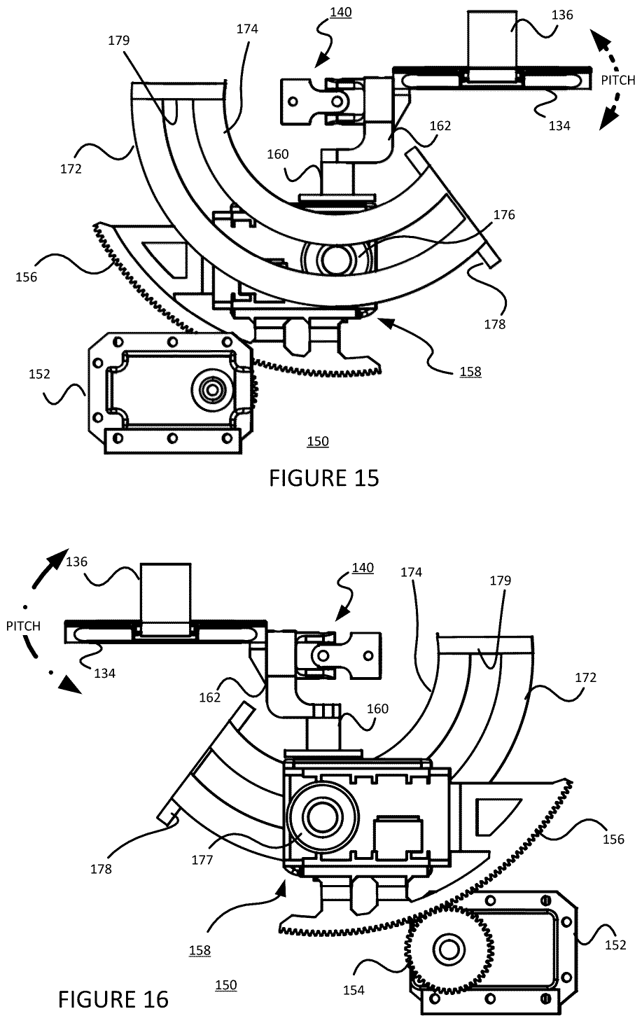

The yaw movement is now described. The second servomotor 158, which is mounted securely (e.g., with a bracket 173 as shown in FIG. 11A) to the second spur gear 156, provides yaw motion. In some example embodiments, this bracket is designed to incorporate the first and second bearings 176, 177 on either side of the second servomotor 158. These bearings 176, 177, which are attached via sturdy guiding rails allow the second servomotor 158 to move along an arc on a plane extending through the front and back of the device 100 during pitch motion. Recall from FIGS. 11B, 11C and 13B that the bearing 177 sits in guide rail(s) 175 that are part of the foot platform (e.g., enclosing chassis) itself. These guide rail(s) 175 guide and provide stability to the yaw motor 158. The second servomotor 158 drives, via shaft 160 and bent arm 162, the toe platform 134 side-to-side along the yaw axis. The guide rails 172, 174, 175 hold the second servomotor 158, via bearings 176, 177 rigidly during its actuation.

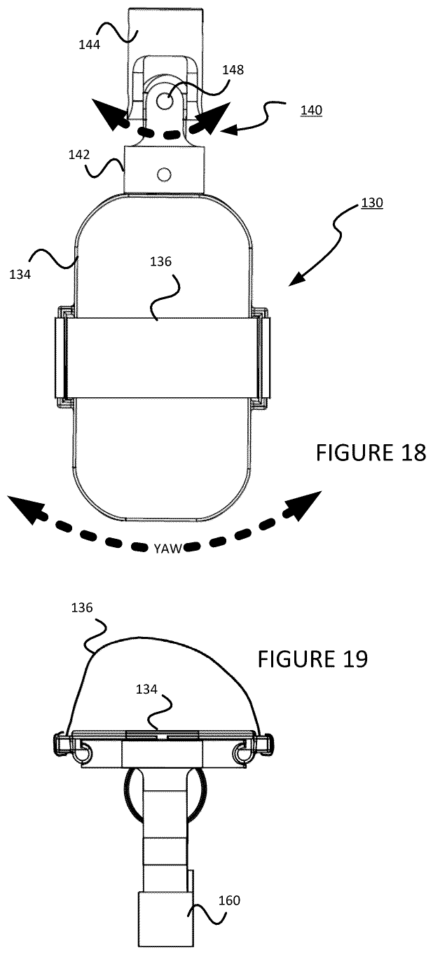

Referring especially to FIGS. 17A-18, the second servomotor 158 is linked, via shaft 160 and bent arm 162 to transmit the yaw movement to the toe platform 134. Note that the yoke/fork attachment 144 of the universal joint 140 is rotatably connected to the main body of the device (e.g., at the vertical wall 127 and/or projection 129), while the yoke/fork extension 142 of the universal joint 140 is attached to, and extends from, the toe platform 134. As the second servomotor 158 is driven, it directly drives, via the shaft 160 and bent arm 162, the universal joint 140, translating the rotation of the shaft 160 of the second servomotor 158 into precise side-to-side adjustments of the toe platform 134, thereby achieving a controlled yaw rotation. Referring to FIG. 17A, note that the shaft 160 is coaxial with the vertical pin 148.

As should be appreciated from the foregoing, the universal joint 140 allows simultaneous movement in both the pitch and yaw directions. Decoupling the two types of motion ensures that the pitch movements driven by the first servomotor 152 do not interfere with the yaw movements driven by the second servomotor 58, and vice-versa. The flexibility of the universal joint 140 allows the toe platform 134 to pivot freely along two axes (defined by the horizontal pin 146 and the vertical pin 148), with each servomotor 152, 158 able to work independently to provide precise control over the orientation of the toe platform 134. In some example embodiments, at any given point of time, only one of the servomotors 152 or 158 actuates while the other servomotor is locked (e.g., electronically), however it should be appreciated that both servomotors 152, 158 may be actuated simultaneously.

To ensure long-term durability and reliable performance, the example turf toe rehabilitation device 100 has a robust mechanical design. For example, the bearing and guide rails system are engineered to reduce (e.g., minimize) friction and wear, and the universal joint 140 provides a secure and stable connection between the moving parts. The bracket holding the second servomotor 158 further enhances this stability, because it can hold the second servomotor 158 in a fixed position during yaw motion while allowing free movement during pitch motion, via the first (e.g., right) bearing 176 along the lower and upper guide rails 172, 174 (and, e.g., via the second (e.g., left) bearing 177 along the additional guide rails, not shown). Additionally, using a gear train for pitch movement allows for precise, fine-tuned control, as gear-driven systems offer high accuracy and torque transfer. The mechanical stops of the second (partial) spur gear 156 are designed to provide a hard, physical limit to the range of pitch motion of the toe platform 134, adding an extra layer of safety and reliability to the example device 100.

The servomotors 152 and 158 are configured to pitch and yaw toe platform assembly 130 as described above. In some example embodiments, servomotors 152 and 158 are configured to provide one or more operating torques in order to drive the respective gears and/or driveshafts. The servomotors 152 and 158 may be configured to provide the same torque, or different torques. The servomotors 152 and 158 may be configured for an active and/or passive mode, as described further herein. In some embodiments, the servomotors 152 and 158 may provide a ramped torque that increases or decreases during operation. A previous study on low-load devices, particularly for the first metatarsophalangeal (MTP) joint, points to an exemplary therapeutic torque range for toe rehabilitation (Buck F. Willis & Sarah A. Curran, “Dynamic Splinting for Contracture Reduction: A Review.” The Foot and Ankle Online Journal 2012; 5 (12): 1.). The study discloses a range of between 0.1 ft-lbs to 2.0 ft-lbs (0.136 Nm to 2.71 Nm). This relates to corresponding force values of between 1.36 N to 27.1 N. In some example embodiments, based on motor torque ranges from 0.01 Nm to 2.0 Nm, and for a toe platform 134 having a length of about 75 mm (e.g., providing a radial arm of approximately 75 mm), toe platform 134 provides about 0.13 N to 26 N force acting on the toe. The disclosed device, servomotors 152,158 and toe platform 134 may operate and/or provide therapy to a subject within any of the disclosed ranges.

In some example embodiments, each servo motor (e.g., servomotors 152, 158) may provide a torque ranging between 0.01 Nm and 2 Nm, between 0.01 Nm and 1 Nm, between 0.01 Nm and 0.5 Nm, between 0.1 Nm and 0.4 Nm, or between 0.2 Nm and 0.3 Nm. In some example embodiments, each servo motor may provide a torque of about 0.05 Nm, 0.1 Nm, 0.15 Nm, 0.2 Nm, 0.25 Nm, 0.3 Nm, 0.35 Nm, 0.4 Nm, 0.45 Nm, 0.5 Nm, 0.55 Nm, 0.6 Nm, 0.65 Nm, 0.7 Nm, 0.75 Nm, 0.8 Nm, 0.85 Nm, 0.9 Nm, 0.95 Nm, 1 Nm, 1.05 Nm, 1.10 Nm, 1.15 Nm, 1.20 Nm, 1.25 Nm, 1.30 Nm, 1.35 Nm, 1.40 Nm, 1.45 Nm, 1.50 Nm, 1.55 Nm, 1.60 Nm, 1.65 Nm, 1.70 Nm, 1.75 Nm, 1.80 Nm, 1.85 Nm, 1.90 Nm, 1.95 Nm, 2.00 Nm, 2.05 Nm, 2.10 Nm, 2.15 Nm, 2.20 Nm, 2.25 Nm, 2.30 Nm, 2.35 Nm, 2.40 Nm, 2.45 Nm, 2.50 Nm, 2.55 Nm, 2.60 Nm, 2.65 Nm, 2.70 Nm, 2.75 Nm, 2.80 Nm, 2.85 Nm, 2.90 Nm, 2.95 Nm, or about 3.00 Nm. In some example embodiments, each servomotor has a torque of about 0.27 Nm. In some example embodiments, toe platform 134 is configured to provide force to a subject's toe ranging between about 0.05 N and about 30 N, about 0.1 N and 28 N, or about 0.15 N and 26 N.

The microcontroller 180 is mounted to the base plate 115, holds software and/or firmware, and communicates with an interface (e.g., smart phone application) either wired or wirelessly, taking the inputs from the user. The interface (e.g., smartphone application) acts as a user interface to allow the user to select modes and/or options. The removable batteries in the battery compartment 190 power the servomotors 152, 158 and/or the microcontroller 180.

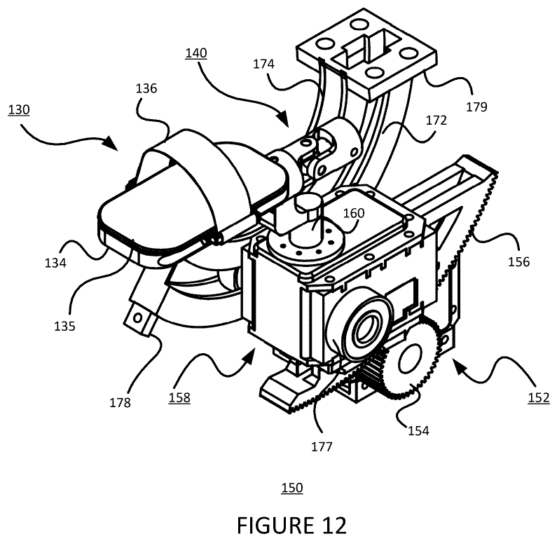

In some example embodiments, the toe platform 134 preferably has a sliding component 135 that sits on top of and is slidably attached to the platform (see FIG. 12). This sliding component 135 allows the toe to slide by a few millimeters as the toe platform moves in the pitch and yaw directions, ensuring safe and seamless movement of the toe as it is strapped in place. In some example embodiments, sliding component 135 is configured to slide in any direction, or is locked to one or more directions (e.g., fore and aft, side to side).

The dimensions of the device will depend on the foot size of the intended user. The device may be sized to align with shoe sizes, or may be produced in various sizes (e.g., small, medium, large). The device may be sized based on the size and shape of the patient's foot and/or big toe. For example, toe platform 134 may have one or more widths ranging between 20 mm and 80 mm, and one or more lengths ranging between 40 mm and 120 mm. In some example embodiments, toe platform 134 has a width of about 40 mm and a length of about 60 mm. In some example embodiments, device 100 has one or more overall lengths ranging between 200 mm and 500 mm, one or more overall widths ranging between 100 mm and 300 mm, and one or more overall heights ranging between 50 mm and 250 mm. In some example embodiments, device 100 has a length of about 340 mm, a width of about 140 mm, and a height of about 125 mm.

As shown throughout the figures, in some example embodiments device 100 comprises a curved or contoured right side wall 124 that follows the contour of a subject's foot, configuring footbed 110 to be in the shape of a subject's foot and/or making a front or distal portion of the device slightly wider than a back or proximal portion of the device. In some example embodiments, device 100 and/or footbed 110 are slanted or angled forward, away from a subject, or slanted or angled backward, toward a subject, however as shown throughout the figures, device 100 may comprise a footbed 110 that is parallel, or substantially parallel to base plate 115. In some embodiments, footbed 110 comprises a raised region for arch support, or a cupped region to hold the heel of the subject. In some example embodiments, the footbed 110 comprises an adjustable cupped region to adjust to the size of the subject's foot to firmly hold the heel and/or foot of the subject. Further, an adjustable side support may also be provided on footbed 110.

Although the example device 100 includes two servomotors—one 152 for pitch rotation and the other 158 for yaw rotation—this is not required. In an alternative design, a single motor can be used to provide both pitch and yaw rotations. When a single motor is used for both pitch and yaw, a mechanism (e.g., a clutch) may be used to selectively power and/or move the toe platform 134 to provide pitch and yaw motion either separately or simultaneously.

Although the example device 100 is battery powered, another power source (e.g., a USB power supply, wall plug and/or AC/DC converter) can be used instead or in addition.

Generally, device 100 (e.g., base plate 115, footbed 110, left and right side walls 122, 124) comprises and/or is formed from any suitably rigid materials. For example, in some example embodiments, the device 100 comprises and/or is formed from inelastic materials, rigid materials, sterilizable materials, biocompatible materials, soft materials, and the like, and any combinations thereof. Example materials include any metals, alloys, foams, gels, composites and plastics (e.g., ABS, PETG, TPU). While ABS offers sufficient strength for the device (e.g., base plate 115, footbed 110, left and right side walls 122, 124), PETG may also be used with its superior chemical resistance and biocompatibility. For any patient-contact surface (e.g., toe platform 134, sliding component 135, footbed 110), a softer, medical-grade material like silicone or TPU may be used for comfort and hygiene.

Strap 136 may comprise inelastic materials and/or elastic materials. In some example embodiments, strap 136 comprises a hook and loop structure. Any portion of device 100 may comprise one or more coatings, such as anti-microbial coating, anti-fungal coating, copper coating, and the like. Portions of device 100 (e.g., footbed 110, toe platform 134) may comprise soft or compressible layers or coatings. For example, portions of device 100 may comprise one or more foam layers. A bottom surface of device 100 (e.g., the bottom surface of base plate 115) may comprise a high friction material and/or coating to prevent device 100 from moving during use.

### § 5.2 EXAMPLE METHOD(S) USING EXAMPLE TURF TOE REHABILITATION DEVICE(S)

In phase one, as the initial pain subsides, regular controlled motion is provided to the main toe by providing pitch and yaw motion in the form of assistive motion with the help of the servomotors. This is achieved by placing the toe on the toe platform, and strapping the toe with a hook and loop strap to constrain the movement. The range of motion can be limited according to the needs of the patient (e.g., per a physical therapy plan provided by a physical therapist or determined automatically (e.g., using artificial intelligence).

For phase two, the patient exerts force to build muscle strength, and hence no active forces need to be added to the main toe. Here, the patient exerts the force on the toe platform in the downward direction. Further, the toe platform is connected to the first servomotor through linkage limiting the range of this downward motion, as discussed above. Resistance is provided to the user with the help of the first servomotor, which must be overcome by the patient. Further, the toe platform has to be pushed down until a certain preset angle, after which it will return to the starting position automatically, or upon receipt of a user-entered command.

The number of repetitions of the rehabilitative cycles for both phases can be recorded and provided to the user, physical therapist, and/or an expert system for reference.

One example embodiment of the device 100 allows for approximately 35° of dorsiflexion and approximately 35° of plantar flexion, which may be sufficient for rehabilitation of an injured toe. Nonetheless, with some redesign, these ranges can potentially be further increased to 70° and 53°, respectively.

A patient in need of toe rehabilitation would place their foot onto the footbed and the toe would lie on top of the toe platform. As can be appreciated from the foregoing description of the example device 100, the two servomotors 152, 158 facilitate the lateral and plantar/dorsiflexion.

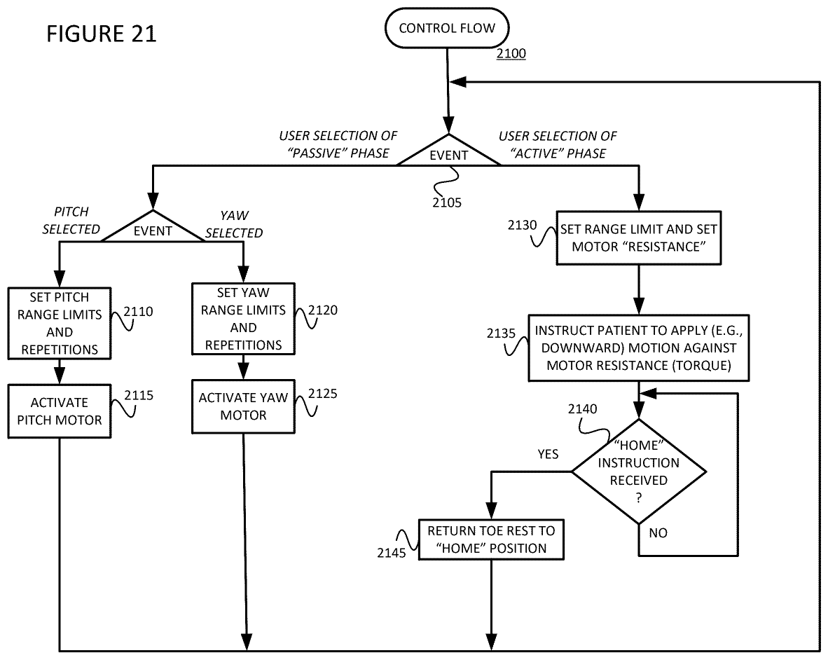

FIG. 21 is a flow diagram of an example method 2100 for using the example toe rehabilitation device with a simple, button-based UI or a more via soft buttons on an application (e.g., on a mobile device). On the start of the program, a display (e.g., an LCD display, or the screen of a mobile device) is initialized and gives a prompt to press either button 1 for Phase 1 (Passive Phase) or Button 2 for Phase 2 (Active Phase). On pressing the button 1 (See left branch from condition 2105), the user interface displays phase one starting in two seconds and prompts the user to press a first button for pitch motion or a second button for yaw motion. On pressing the first button, pitch motion is chosen and range limits and/or a number of repetitions is set (Block 2110). Then, the pitch motor is activated to perform the set number (e.g., 3) of pitch motion repetitions starting from its home position over the set range (e.g., for +35° to −35°) (Block 2115). After completion of this, the user interface display prompts the user again for pitch and yaw motion pressing the second button. Again, range limits and/or a number of repetitions is set (Block 2120). Then, the yaw motor is activated to perform the set number of (e.g., 3) repetitions over the set range limit (e.g., 30 degrees to the left) (Block 2125).

In Phase 2 (See the right branch from event 2105.), the range limit and amount of motor resistance are set (Block 2130). Then, the user interface display is used to instruct the patient to push the platform downwards and to press a third button after the desired position is obtained (Block 2135). Once the third button is pressed (Decision 2140=YES), the pitch motor moves the platform to the home position (Block 2145), and the process is repeated for the set number of (e.g., 3) repetitions. This completes both active and passive rehabilitation and user this prompted again to start with Phase one or Phase two. The buttons may be physical buttons, or soft buttons displayed on a touch display. After Completion of both pitch and yaw motions, the user is prompted again to select Phase 1 (Passive) or Phase 2 (Active).

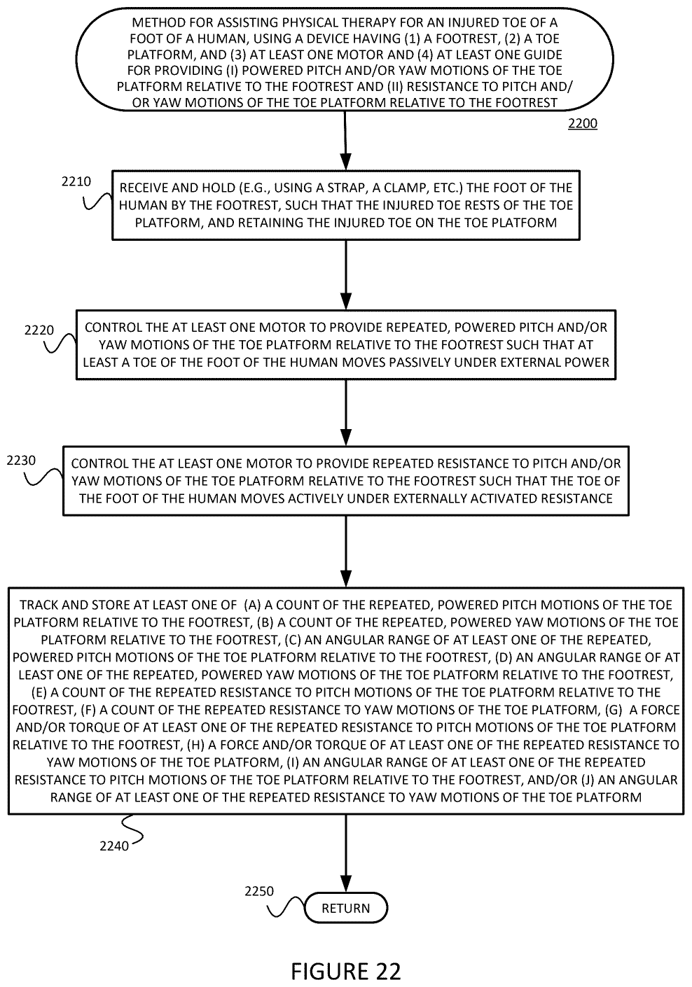

FIG. 22 is a more generic method 2200 for assisting physical therapy for an injured toe of a foot of a human, using a device having a footbed (Recall, e.g., 110.), a toe platform (Recall, e.g., 134), at least one motor (Recall, e.g., servomotors 152 and 158,) and at least one guide (Recall, e.g., guide rails 172 and 174) for providing (i) powered pitch and/or yaw motions of the toe platform relative to the footrest and (ii) resistance to pitch and/or yaw motions of the toe platform relative to the footrest. As shown, this example method 2200 includes:
receiving and holding (e.g., using a strap, a clamp, etc.) the foot of the human by the footrest, such that the injured toe rests of the toe platform, and retaining the injured toe on the toe platform (Block 2210);
controlling the at least one motor to provide repeated, powered pitch and/or yaw motions of the toe platform relative to the footrest such that at least a toe of the foot of the human moves passively under external power (Block 2220); and
controlling the at least one motor to provide repeated resistance to pitch and/or yaw motions of the toe platform relative to the footrest such that the toe of the foot of the human moves actively under externally activated resistance (Block 2230).

The example method 2200 may further include tracking and storing at least one of (A) a count of the repeated, powered pitch motions of the toe platform relative to the footrest, (B) a count of the repeated, powered yaw motions of the toe platform relative to the footrest, (C) an angular range of at least one of the repeated, powered pitch motions of the toe platform relative to the footrest, (D) an angular range of at least one of the repeated, powered yaw motions of the toe platform relative to the footrest, (E) a count of the repeated resistance to pitch motions of the toe platform relative to the footrest, (F) a count of the repeated resistance to yaw motions of the toe platform, (G) a force and/or torque of at least one of the repeated resistance to pitch motions of the toe platform relative to the footrest, (H) a force and/or torque of at least one of the repeated resistance to yaw motions of the toe platform, (I) an angular range of at least one of the repeated resistance to pitch motions of the toe platform relative to the footrest, and/or (J) an angular range of at least one of the repeated resistance to yaw motions of the toe platform (Block 2240).

These steps may be repeated, with the same or different parameters, at (e.g., prescribed) intervals. Although methods and steps disclosed in this application are ordered alphanumerically, the steps may be performed in any order, and each step may be performed any number of times.

Referring back to block 2230, in some example embodiments, the act of controlling the at least one (e.g., two) motor to provide repeated, powered pitch and/or yaw motions of the toe platform relative to the footrest, limits the pitch and/or yaw motions to angular limits. In one example, the angular limits are increased over time (e.g., such that the limits are constrained to be within the normative range of pitch and/or yaw motion of human toe, e.g., starting with a fraction of the normative range, and increased slowly to reach the normative range or values according to a treatment protocol prescribed by the therapist). More generally, the angular limits may be adjusted (e.g., adapted by the therapist) based on express or implied patient feedback, the speed of recovery of patient, reinjury, setback, etc. For example, the patient can lower, via a user interface, the angular limits and/or angular speed of pitch and yaw movements (e.g., in case of feeling of discomfort).

Referring back to block 2220, in at least one example method, the act of controlling the at least one motor to provide repeated resistance to pitch and/or yaw motions of the toe platform relative to the footrest, increases the resistance over time. This may be done, for example, by adapting the torque resistance offered by the at least one motor.

In some aspects of the present invention, software executing the instructions provided herein may be stored on a non-transitory computer-readable medium, wherein the software performs some or all of the steps of the present invention when executed on a processor.

Aspects of the invention relate to algorithms executed in computer software. Though certain embodiments may be described as written in particular programming languages, or executed on particular operating systems or computing platforms, it is understood that the system and method of the present invention is not limited to any particular computing language, platform, or combination thereof. Software executing the algorithms described herein may be written in any programming language known in the art, compiled, or interpreted, including but not limited to C, C++, C#, Objective-C, Java, JavaScript, MATLAB, Python, PHP, Perl, Ruby, or Visual Basic. It is further understood that elements of the present invention may be executed on any acceptable computing platform, including but not limited to a server, a cloud instance, a workstation, a thin client, a mobile device, an embedded microcontroller, a television, or any other suitable computing device known in the art.

Parts of this invention are described as software running on a computing device. Though software described herein may be disclosed as operating on one particular computing device (e.g. a dedicated server or a workstation), it is understood in the art that software is intrinsically portable and that most software running on a dedicated server may also be run, for the purposes of the present invention, on any of a wide range of devices including desktop or mobile devices, laptops, tablets, smartphones, watches, wearable electronics or other wireless digital/cellular phones, televisions, cloud instances, embedded microcontrollers, thin client devices, or any other suitable computing device known in the art. Example embodiments consistent with the present description might be implemented in hardware, such as one or more field programmable gate arrays (“FPGA”s), one or more integrated circuits such as ASICs, one or more network processors, etc.

Similarly, parts of this invention are described as communicating over a variety of wireless or wired computer networks. For the purposes of this invention, the words “network”, “networked”, and “networking” are understood to encompass wired Ethernet, fiber optic connections, wireless connections including any of the various 802.11 standards, cellular WAN infrastructures such as 3G, 4G/LTE, or 5G networks, Bluetooth®, Bluetooth® Low Energy (BLE) or Zigbee® communication links, or any other method by which one electronic device is capable of communicating with another. In some embodiments, elements of the networked portion of the invention may be implemented over a Virtual Private Network (VPN).

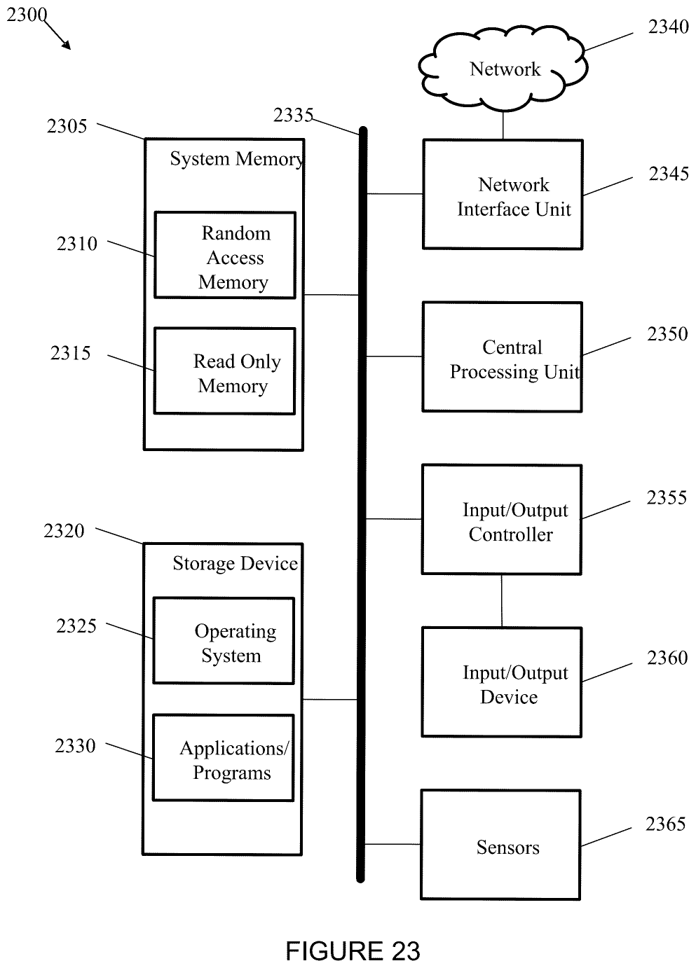

FIG. 23 and the following discussion are intended to provide a brief, general description of a suitable computing environment in which the invention may be implemented. While the invention is described above in the general context of program modules that execute in conjunction with an application program that runs on an operating system on a computer, those skilled in the art will recognize that the invention may also be implemented in combination with other program modules.

Generally, program modules include routines, programs, components, data structures, and other types of structures that perform particular tasks or implement particular abstract data types. Moreover, those skilled in the art will appreciate that the invention may be practiced with other computer system configurations, including hand-held devices, multiprocessor systems, microprocessor-based or programmable consumer electronics, minicomputers, mainframe computers, and the like. The invention may also be practiced in distributed computing environments where tasks are performed by remote processing devices that are linked through a communications network. In a distributed computing environment, program modules may be located in both local and remote memory storage devices.

FIG. 23 depicts an illustrative computer architecture for a computer 2300 for practicing the various embodiments of the invention. The computer architecture shown in FIG. 23 illustrates a conventional personal computer, including a central processing unit 2350 (“CPU”), a system memory 2305, including a random access memory 2310 (“RAM”) and a read-only memory (“ROM”) 2315, and a system bus 2335 that couples the system memory 2305 to the CPU 2350. A basic input/output system containing the basic routines that help to transfer information between elements within the computer, such as during startup, is stored in the ROM 2315. The computer 2300 further includes a storage device 2320 for storing an operating system 2325, application/program 2330, and data.

The storage device 2320 is connected to the CPU 2350 through a storage controller (not shown) connected to the bus 2335. The storage device 2320 and its associated computer-readable media provide non-volatile storage for the computer 2300. Although the description of computer-readable media contained herein refers to a storage device, such as a hard disk or CD-ROM drive, it should be appreciated by those skilled in the art that computer-readable media can be any available media that can be accessed by the computer 2300.

By way of example, and not to be limiting, computer-readable media may comprise computer storage media. Computer storage media includes volatile and non-volatile, removable and non-removable media implemented in any method or technology for storage of information such as computer-readable instructions, data structures, program modules or other data. Computer storage media includes, but is not limited to, RAM, ROM, EPROM, EEPROM, flash memory or other solid state memory technology, CD-ROM, DVD, or other optical storage, magnetic cassettes, magnetic tape, magnetic disk storage or other magnetic storage devices, or any other medium which can be used to store the desired information and which can be accessed by the computer.

According to various embodiments of the invention, the computer 2300 may operate in a networked environment using logical connections to remote computers through a network 2340, such as TCP/IP network such as the Internet or an intranet. The computer 2300 may connect to the network 2340 through a network interface unit 2345 connected to the bus 2335. It should be appreciated that the network interface unit 2345 may also be utilized to connect to other types of networks and remote computer systems.

The computer 2300 may also include an input/output controller 2355 for receiving and processing input from a number of input/output devices 2360, including a keyboard, a mouse, a touchscreen, a camera, a microphone, a controller, a joystick, or other type of input device. Similarly, the input/output controller 2355 may provide output to a display screen, a printer, a speaker, or other type of output device. The computer 2300 can connect to the input/output device 2360 via a wired connection including, but not limited to, fiber optic, Ethernet, or copper wire or wireless means including, but not limited to, Wi-Fi, Bluetooth, Near-Field Communication (NFC), infrared, or other suitable wired or wireless connections.

As mentioned briefly above, a number of program modules and data files may be stored in the storage device 2320 and/or RAM 2310 of the computer 2300, including an operating system 2325 suitable for controlling the operation of a networked computer. The storage device 2320 and RAM 2310 may also store one or more applications/programs 2330. In particular, the storage device 2320 and RAM 2310 may store an application/program 2330 for providing a variety of functionalities to a user. For instance, the application/program 2330 may comprise many types of programs such as a word processing application, a spreadsheet application, a desktop publishing application, a database application, a gaming application, internet browsing application, electronic mail application, messaging application, and the like. According to an embodiment of the present invention, the application/program 2330 comprises a multiple functionality software application for providing word processing functionality, slide presentation functionality, spreadsheet functionality, database functionality and the like.

The computer 2300 in some embodiments can include a variety of sensors 2365 for capturing measurements from any disclosed device (e.g., device 100), monitoring the environment surrounding the device, and a monitoring or capturing subject measurements. These sensors 2365 can include a Global Positioning System (GPS) sensor, a photosensitive sensor, a camera, a gyroscope, a magnetometer, a position sensor, a position and/or rotation encoder, a force sensor, a torque sensor, a load sensor, a thermometer, a proximity sensor, an accelerometer, an inertial measurement unit (IMU), a motion sensor, a microphone, a biometric sensor, a barometer, a humidity sensor, a radiation sensor, or any other suitable sensor.

It should be appreciated that the disclosed sensors may be used to measure any portion of device 100, such as position, force and/or torque sensors measuring the position of toe platform 134 and/or servomotors 152, 158. In some example embodiments, a pressure sensor, proximity sensor and/or a photosensitive sensor is positioned on footbed 110 and/or toe platform 134 to detect the presence or absence of a subject's foot or toe, respectively. The presence or absence of a subject's foot or toe may be used to actuate or deactivate the device, and enable or cease any methods disclosed herein. In some example embodiments, a pressure sensor is positioned on toe platform 134 to detect the pressure of the subject's toe against the platform which may be used to enable or cease motion of the toe platform 134 if a threshold is exceeded. For example, if a pressure exceeds an upper bound of a threshold, motion of the toe platform 134 may cease to prevent injury to the subject.

The disclosed device and methods of use thereof may operate at least in part with a user interface (UI) and/or graphical user interface (GUI) that may at least partially reside on a computing device, such as computer 2300 disclosed herein. It should be appreciated that any parts, modules, and/or features of the disclosed systems and methods may be represented on a physical interface comprising a UI and/or a display with a GUI, either as physical or graphical buttons. Further, the inputs and outputs of the systems may be displayed and/or produced via the UI and/or GUI, either as data or messages, analog sounds produced by the system itself, digital representations of sounds produced by the system, graphical representations of sounds on a display, and/or LEDs representative of sounds, messages, data, and the like. In some example embodiments, an interface comprises a display mounted on device 100 displaying a GUI. In other example embodiments, a GUI comprises an application on a handheld device, as discussed further herein.

Further, the disclosed device and associated methods may operate, at least in part, with one or more artificial intelligence (AI) models and/or machine learning (ML) algorithms, engines, and modules that may reside on a computing device (e.g., computer 2300 disclosed herein). These models may function in conjunction with one or more networks (e.g., neural networks) to capture and analyze subject-specific data derived from the device's sensors and functional components. In some embodiments, the AI/ML system is designed to learn individualized aspects of the subject and dynamically modulate the device's functions and/or treatment methods. For example, the system may measure or aggregate data such as biometrics, body size, weight, foot anatomy, toe strength, injury type, prior treatments, ongoing therapy sessions, and planned future interventions. This information may be used to train a neural network that modulates or adjusts the device's outputs—such as assistive motion, applied forces, and torques generated by the toe platform 134 and/or servomotors 152, 158—based on real-time and historical data. In some embodiments, AI/ML may also provide individualized treatment recommendations and/or predict treatment outcomes based on subject-specific data and injury profiles. In further embodiments, the system may generate recovery plans and forecast recovery timelines tailored to the subject's physiology and injury progression.

In some embodiments, the AI/ML engine may operate in real time to analyze feedback from embedded sensors, such as force sensors, encoders, inertial measurement units (IMUs), or pressure sensors, to assess subject compliance, muscle activation, joint articulation, and load distribution. Based on these inputs, the system may intelligently adjust rehabilitation protocols during a single therapy session—for instance, by reducing force output if excessive resistance is detected or increasing range of motion as mobility improves. Additionally, the system may detect patterns associated with fatigue, compensatory movement, or pain avoidance behavior and intervene accordingly, such as triggering real-time alerts or modifying parameters to ensure safety and optimal therapeutic outcomes.

In some example embodiments, an AI/ML module may perform longitudinal tracking of subject performance and rehabilitation progress over multiple sessions. By analyzing trends in force output, joint angles, movement smoothness, compliance, and session frequency, the system can predict recovery trajectories, identify plateaus, and offer adaptive treatment plans that evolve over time. Predictive models may also estimate when a patient is likely to regain specific functional capabilities (e.g., return to sport or pain-free walking) and flag deviations from expected progress that may indicate the need for clinical reassessment or alternative interventions. These insights may be visualized through dashboards or reports accessible to clinicians or users.

In some embodiments, anonymized user data may be aggregated in a cloud-based infrastructure to enable population-level analysis and continuous improvement of the AI/ML models via federated or distributed learning. Such configurations allow the system to learn from a broad spectrum of injury types, recovery responses, and demographic variables, which in turn enhances the predictive accuracy and personalization capabilities for new or existing subjects. Furthermore, cloud connectivity can support remote monitoring by clinicians, who may receive alerts regarding patient non-compliance, abnormal recovery patterns, or unexpected biomechanical responses. This connectivity also facilitates tele-rehabilitation by enabling clinicians to update therapy plans remotely based on AI-derived insights.

Dynamic Resistance Profiling (Reinforcement Learning—RL): In some example embodiments, an RL agent can learn the optimal assistance/resistance level for each movement. The goal would be to maximize therapeutic effectiveness (e.g., completing full range of motion) while minimizing indicators of pain or fatigue (e.g., jerky movements, incomplete reps), thus adapting each session to the user's daily condition. Predictive Progression Modeling (Regression): In some example embodiments, a regression model can be trained on session data to predict the maximum safe range of motion for the next session. This would automate the progression of treatment.

In certain embodiments, the device operates as a closed-loop adaptive feedback system wherein real-time sensor data is continuously analyzed by one or more AI/ML models to autonomously modify therapeutic parameters without clinician intervention. This feedback loop may include input signals from biomechanical sensors (e.g., load sensors, torque sensors, or motion trackers), which are processed through predictive or reinforcement learning algorithms trained to identify optimal treatment responses. The AI system may compute adjustments to motor output, toe platform trajectory, or resistance levels in direct response to subject-specific performance metrics (e.g., insufficient toe extension, abnormal force curve, delayed response time). This integrated and automated control loop enables the device to deliver dynamically personalized rehabilitation in a way that adapts in real time to patient progress, fatigue, or pain-related inhibition-providing a significant improvement over static, clinician-set protocols and enhancing treatment efficacy, safety, and user adherence.

### § 5.3 ALTERNATIVES, EXTENSIONS, AND/OR REFINEMENTS

### § 5.3.1 Example Motor(S)

In one example embodiment of the example device 100, two standard servomotors from Parallax are used, one for the pitch motion the other for yaw motion. Each of these servomotors can hold any position over a 180-degree range and has a torque of 0.27 Nm at 6V. Higher torque motors from Dynamixel may be used instead.

A more sophisticated motor may be used to allow for fine torque control to cater for an individual's rehabilitation requirements, such as varying required torque in active rehabilitation process. In addition, the device interface may be simplified by making an easy-to-navigate application which can be used by the user to operate the device. This will also allow the device to be mobile and wireless, making it more portable.

### § 5.3.2 Example Controller(S)

In some example embodiment of the example device 100, the microcontroller 180 is an Arduino Uno. It is an open-source microcontroller, and can be powered by a USB connection. Alternatively, or in addition, it may be battery powered. It gathers the information from the sensor (e.g., selection push buttons discussed below) and the user can reprogram accordingly. All the data from the sensors are obtained by the Arduino to perform the task accordingly. Other programmable controllers and/or hardware can be used instead.

### § 5.3.3 Example User Interfaces

### § 5.3.3.1 Example Button-Based User Interface

Push button(s) can be used for selection of Phases when prompted. For example, first and second push buttons can be used as selector buttons, and a third push button can be used in Phase 2 of the program.

### § 5.3.3.2 Example App-Based User Interface (UI)

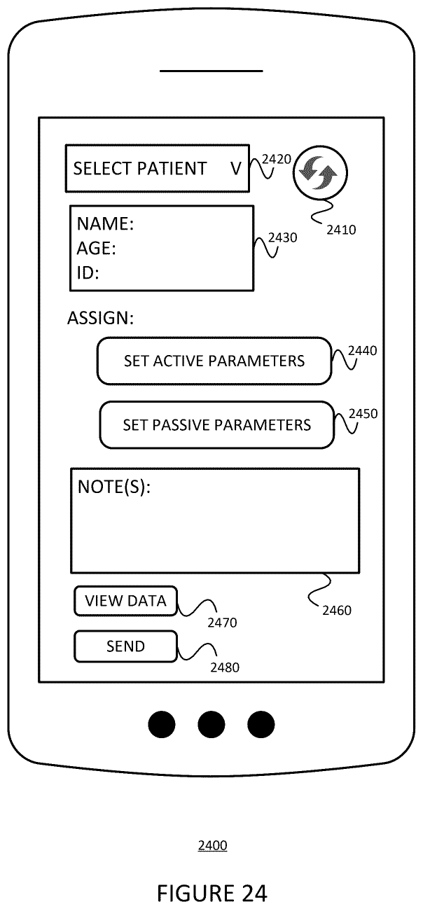

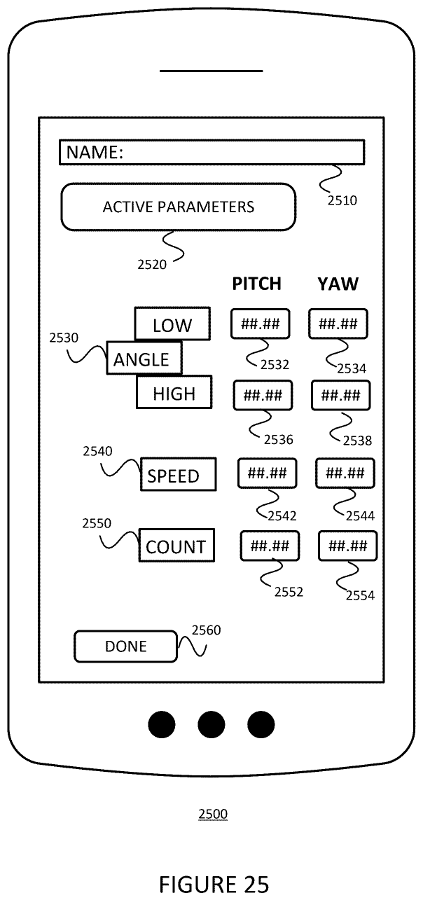

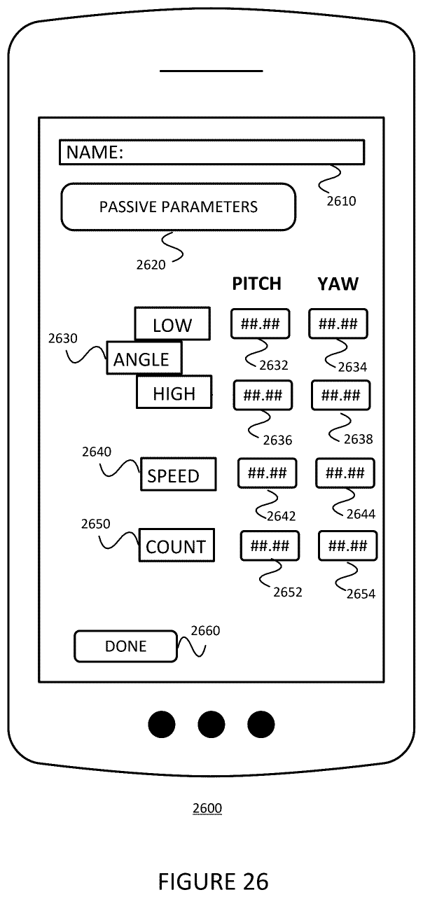

FIGS. 24-26 illustrate an example Turf-Toe Rehabilitation Device User Interface (UI) on therapist's side. These example UIs may facilitate the assignment of the treatment parameters and monitoring. For example, referring to example user interface screen 2400, using a dropdown menu 2420, a therapist can select a specific user's device. The patient's information is displayed in section 2430. The therapist may then assign either parameters for active or passive (resistive) therapy using soft buttons 2440 and 2450, respectively. Notes may be entered in area 2460. Previous data may be viewed using soft button 2470. Soft button 2480 may be used to send data (e.g., entered parameters) to an external device.

Referring to user interface display 2500, based on the treatment plan for the selected patient (whose name is displayed in section 2510), the therapist may then assign the angle 2530, speed 2540, and count 2550 for pitch and/or yaw in the active phase. In this example, the fact that the screen 2500 concerns active parameters is indicated by display section 2520. Low pitch and yaw angles are entered in areas 2532 and 2534, respectively. Similarly, high pitch and yaw angels are entered in areas 2536 and 2538, respectively. Speed for pitch and yaw are entered in areas 2542 and 2544, respectively. Finally, counts for pitch and yaw are entered in areas 2552 and 2554, respectively. The therapist can add a note to alert the user while sending the new parameters to the user.

Similarly, referring to user interface display 2600, based on the treatment plan for the selected patient (whose name is displayed in section 2610), the therapist may then assign the angle 2630, speed 2640, and count 2650 for pitch and/or yaw in the passive phase. In this example, the fact that the screen 2600 concerns passive parameters is indicated by display section 2620. Low pitch and yaw angles are entered in areas 2632 and 2634, respectively. Similarly, high pitch and yaw angels are entered in areas 2636 and 2638, respectively. Speed for pitch and yaw are entered in areas 2642 and 2644, respectively. Finally, counts for pitch and yar are entered in areas 2652 and 2654, respectively. The therapist can add a note to alert the user while sending the new parameters to the user.

Referring back to FIG. 24, the example UI display 2400 has a refresh button 2410 to retrieve information forcibly. The therapist can review the user's past assigned exercises and monitor the exercises for any overrides in case of pain or other emergencies. The therapist can download the data in an Excel file to process information in graphs and tables and can assign future exercises based on the history.

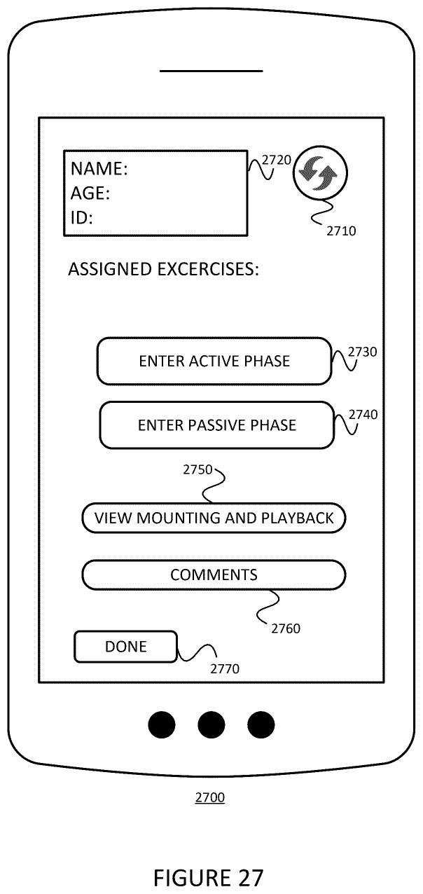

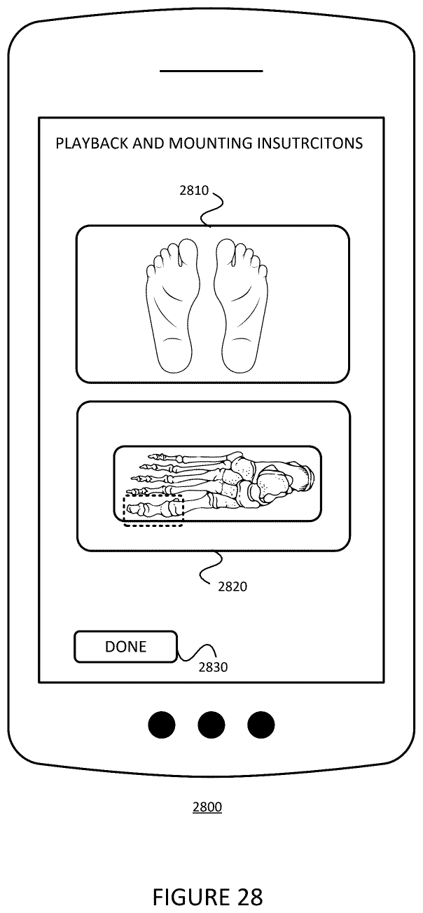

FIGS. 27 and 28 illustrate example UI displays on the patient's side. These example UI displays 2700 and 2800 may be used to receive parameter updates and/or performing exercises. For example, referring to FIG. 27, after receiving data from the therapist, using the UI, the user can choose between active or passive treatment using soft buttons 2730 and 2740. Further, the UI can allow the user to select whether to perform a pitch or yaw exercise (using further soft buttons, not shown). The user can also use soft button 2750 to view the procedure for mounting their foot and toe. Areas 2810 and 2820 of the example user interface display 2800 of FIG. 28 provide display areas using images and/or videos to illustrate the mounting and/or exercise suggested by their therapist.

Once the user is ready, they can begin the exercise and pause or stop if necessary in case of an emergency. In the event of pain, the user can use the UI to click on “indicate pain and override” to override the exercise. Using the UI, the user can also decrease the parameters and continue the exercise. After completion, the user can leave comments via soft button 2760 and click “done” via soft button 2770 or 2830. Any overrides can be automatically recorded and included in the data being sent to the therapist by clicking the “done” soft button.

The user interface can be further adapted using guidance from Bethi et al. (See, e.g., the article, S. R. Bethi, A. RajKumar, F. Vulpi, P. Raghavan and V. Kapila, “Wearable Inertial Sensors for Exergames and Rehabilitation,” 2020 42nd Annual International Conference of the IEEE Engineering in Medicine & Biology Society (EMBC), Montreal, QC, Canada, 2020, pp. 4579-4582, doi: 10.1109/EMBC44109.2020.9175428, incorporated herein by reference.)

### § 5.4 CONCLUSIONS

Example embodiments and methods consistent with the present description facilitate effective and safe turf toe rehabilitation by patients and their therapists.

What is claimed is:

1. A method for assisting physical therapy for an injured toe of a foot of a human, using a device having a footrest;
a toe platform; and
at least one motor and at least one guide for providing at least one of (i) powered pitch and/or yaw motions of the toe platform relative to the footrest, and (ii) resistance to pitch and/or yaw motions of the toe platform relative to the footrest, the method comprising:
receiving and holding the foot of the human by the footrest, such that the injured toe rests of the toe platform, and retaining the injured toe on the toe platform;
controlling the at least one motor to provide repeated, powered pitch and/or yaw motions of the toe platform relative to the footrest such that at least a toe of the foot of the human moves passively under external power; and
controlling the at least one motor to provide repeated resistance to pitch and/or yaw motions of the toe platform relative to the footrest such that the toe of the foot of the human moves actively under externally activated resistance.

2. The method of claim 1, further comprising, after controlling the at least one motor to provide repeated, powered pitch and/or yaw motions of the toe platform relative to the footrest but before controlling the at least one motor to provide repeated resistance to pitch and/or yaw motions of the toe platform relative to the footrest:
removing the foot of the human from the footrest and the injured toe from the toe platform;
after waiting for at least a prescribed rest period between passive and active movements of toe, as prescribed by a therapist based on injury and therapy protocol,
receiving and holding the foot of the human by the footrest such that the injured toe rests on the toe platform, and retaining the injured toe on the toe platform.

3. The method of claim 1, wherein the act of controlling the at least one motor to provide repeated, powered pitch and/or yaw motions of the toe platform relative to the footrest, limits the pitch and/or yaw motions to angular limits.

4. The method of claim 3, wherein the angular limits are increased over time, but such that the limits are constrained to be within the normative range of pitch and/or yaw motion of human toe.

5. The method of claim 3, wherein the angular limits are adjusted based on at least one of express patient feedback, implied patient feedback, recovery history of patient, reinjury, and rehabilitation setback.

6. The method of claim 5, wherein the patient can lower, via a user interface, at least one of the angular limits and angular speed of pitch and yaw movements.

7. The method of claim 1, wherein the act of controlling the at least one motor to provide repeated resistance to at least one of pitch and yaw motions of the toe platform relative to the footrest, increases the resistance over time, and
wherein the resistance is increased over time by adapting the torque resistance offered by the at least one motor.

8. The method of claim 1, further comprising:
tracking and storing at least one of: a count of the repeated, powered pitch motions of the toe platform relative to the footrest, a count of the repeated, powered yaw motions of the toe platform relative to the footrest, an angular range of at least one of the repeated, powered pitch motions of the toe platform relative to the footrest, an angular range of at least one of the repeated, powered yaw motions of the toe platform relative to the footrest, a count of the repeated resistance to pitch motions of the toe platform relative to the footrest, a count of the repeated resistance to yaw motions of the toe platform, a force and/or torque of at least one of the repeated resistance to pitch motions of the toe platform relative to the footrest, a force and/or torque of at least one of the repeated resistance to yaw motions of the toe platform, an angular range of at least one of the repeated resistance to pitch motions of the toe platform relative to the footrest, and an angular range of at least one of the repeated resistance to yaw motions of the toe platform.

9. The method of claim 8, further comprising:
processing at least a portion of the tracked and stored data using one or more artificial intelligence (AI) and/or machine learning (ML) models trained to identify subject-specific rehabilitation parameters; and
adjusting at least one of a powered motion or a resistance profile of the toe platform in real time based on output from the AI and/or ML models to personalize treatment for the injured toe.

10. A device for assisting physical therapy for an injured toe of a foot of a human, the device comprising:
a footrest;
a toe platform;
a motorized assembly mechanically coupled with the toe platform;
guide rails; and
a controller configured to control the motorized assembly to provide, in cooperation with the guide rails, at least one of:
(i) powered pitch and/or yaw motions, either individually or simultaneously, of the toe platform relative to the footrest, and
(ii) resistance to pitch and/or yaw motions, either individually or simultaneously, of the toe platform relative to the footrest.

11. The device of claim 10 further comprising:
a universal joint mechanically coupling the footrest and the toe platform.

12. The device of claim 10, wherein the motorized assembly includes
a first motor for providing, via a gear train, at least one of: (i) powered pitch motions of the toe platform relative to the footrest, and (ii) resistance to pitch motions of the toe platform relative to the footrest, and
a second motor for providing at least one of (i) powered yaw motions of the toe platform relative to the footrest, and (ii) resistance to yaw motions of the toe platform relative to the footrest.

13. The device of claim 12, wherein the controller controls each of the first and second motors to limit the pitch and/or yaw motions to angular limits, either individually or simultaneously.

14. The device of claim 13, wherein the angular limits are increased over time, but such that the limits are constrained to be within the normative range of pitch and/or yaw motion of human toe.

15. The device of claim 10, further comprising:
a physical or soft button for receiving therapist and/or patient feedback, wherein the means for receiving therapist and/or patient feedback provides the feedback to the controller to control at least one of: range of motion of yaw angle, range of motion of pitch angle, resistance in the yaw direction, and/or resistance in the pitch direction.

16. The device of claim 10, wherein the controller is further configured to track and store at least one of: a count of the repeated, powered pitch motions of the toe platform relative to the footrest, a count of the repeated, powered yaw motions of the toe platform relative to the footrest, an angular range of at least one of the repeated, powered pitch motions of the toe platform relative to the footrest, an angular range of at least one of the repeated, powered yaw motions of the toe platform relative to the footrest, a count of the repeated resistance to pitch motions of the toe platform relative to the footrest, a count of the repeated resistance to yaw motions of the toe platform, a force of at least one of the repeated resistance to pitch motions of the toe platform relative to the footrest, a force of at least one of the repeated resistance to yaw motions of the toe platform, an angular range of at least one of the repeated resistance to pitch motions of the toe platform relative to the footrest, and/or an angular range of at least one of the repeated resistance to yaw motions of the toe platform.

17. The device of claim 16, wherein information tracked by the controller is stored on at least one of a non-transitory computer-readable storage medium on the device, and/or a non-transitory computer-readable storage medium on a mobile device in wireless communication with the device.

18. The device of claim 10, wherein the controller is further configured to: receive a user input selecting a first or second phase of physical therapy, and select a first phase controlling the at least one motor to provide repeated, powered pitch and/or yaw motions of the toe platform relative to the footrest responsive to receiving a user input selection of the first phase of physical therapy, or select a second phase controlling the at least one motor to provide repeated resistance to pitch and/or yaw motions of the toe platform relative to the footrest responsive to receiving a user input selection of the second phase of physical therapy.

19. The device of claim 18, wherein the pitch has a maximum value of either approximately 35 degrees or approximately −35 degrees.

20. The device of claim 18, wherein a range of motion of the yaw has a maximum value of approximately 30 degrees.
